(19) United States
(12) Patent Application Publication
(10) Pub. No.: US 2003/0033635 A1
(43) Pub. Date: Feb. 13, 2003

(54) Self-excising polynucleotides and uses thereof

(76) Inventors: Luke Mankin, Raleigh, NC (US); Bryan McKersie, Apex, NC (US)

Correspondence Address: SUTHERLAND ASBILL & BRENNAN LLP, 999 PEACHTREE STREET, N.E., ATLANTA, GA, 30309, US

(21) Appl. No.: 09/940,550

(22) Filed: Aug. 27, 2001

Related U.S. Application Data
(60) Provisional application No. 60/227,961, filed on Aug. 25, 2000.

(51) Int. Cl. A01H 1/00; C12Q 1/68; C07H 21/04; C12N 9/24; C12P 21/02; C12N 5/04

(52) U.S. Cl. 800/287; 435/6; 435/69.1; 435/200; 435/419; 435/320.1; 536/23.6

(57) ABSTRACT

The present invention includes compositions and methods for providing organisms from which transgenic traits can be easily excised. More specifically, the present invention provides self-excising polynucleotides that contain a desired trait and a recombinase polynucleotide. The present invention provides methods for the elimination of unwanted nucleic acids in agricultural food products. Additionally, the compositions and methods of the present invention provide a means to prevent the escape of certain transgenic traits into the environment.

### CROSS REFERENCE TO RELATED APPLICATIONS

This application claims the priority benefit of U.S. Provisional Patent Application Ser. No. 60/227,961 filed Aug. 25, 2000.

### FIELD OF THE INVENTION

This invention generally relates to self-excising recombinant nucleic acids and uses thereof where one can construct a transgenic organism, preferably a plant that contains a nucleic acid that can effectively self-excise under certain conditions.

### BACKGROUND

The incorporation of nucleic acids into plant cells and the subsequent regeneration of fertile plants have enabled the creation of novel phenotypes in a wide range of crops. The benefits of transgenic crops include herbicide resistance, insect resistance and improved nutritional quality traits that have been developed in maize, soybean, rapeseed, tomato, potato, cotton and a wide range of other plant species. The development of crop plants with improved productivity in adverse environments is an additional trait that is currently being developed. However, the escape of certain transgenic traits into the environment and the presence of unwanted nucleic acids in food have raised concerns about the use of recombinant nucleic acid technology in agriculture. It is therefore desirable to remove some or all of the transgenic nucleic acids from crops, especially food commodities, and to restrict the distribution of transgenes within the environment.

A broad class of enzymes, including recombinases, integrases, and translocases, mediates the restriction and excision of nucleic acid sequences. Site-specific recombinases, such as Flp and Cre of the integrase family and φC31 of the recombinase family, recognize specific DNA sequences and perform specific recombinations of the nucleic acid sequence by restricting and ligating the nucleic acid at these sites. The placement and orientation of these target sites determines the type of recombination that occurs. In particular, directly oriented target sites facilitate excision of intervening DNA in the presence of recombinase (Kilby et al. 1995 Plant Journal 8:637-652; Kilby et al. 1993 Trends in Genetics 9:413-421; Pósfai et al. 1994 Nucleic Acids Research 22:2392-2398; Stark et al. 1992 Trends in Genetics 8:432-439).

Site-specific recombinase genes, including Flp, Cre and φC31, are used as tools for genetic engineering because of their simplicity and precision. Flp and Cre recombinase have been successfully employed in a broad range of organisms, including plants (Reviewed by Kilby et al. 1993 Trends in Genetics 9:413-421). The Flp and Cre recombinases recognize distinct 34 bp sites, called FRT and loxP, respectively; the large size and structure of these sites make random occurrences in higher eukaryotic genomes unlikely (Dymecki 1996 PNAS 93:6191-6196, Senacoff et al. 1988 Journal of Molecular Biology 201:405-421), but nonetheless, their use for mutagenesis has been proposed. A more useful irreversible recombination system described in the prior art is the Streptomyces phage φC31 recombination system. A 68 kDa integrase protein recombines an attB site with an attP site. These sites share only three base pairs of homology at the point of cross-over. This homology is flanked by inverted repeats, presumably binding sites for the integrase protein. The minimal known functional size for both the φC31 attB and attP is approximately 30 to 40 base pairs. Unlike other recombinase systems, the φC31 integration reaction is simple in that it does not require a host factor.

Recombinases have been utilized for a diverse set of applications in transgenic plants including control of transgene expression by the excision of blocker DNA by a recombinase resulting in the activation of gene expression (U.S. Pat. No. 5,723,765). Recombinases have also been used to stimulate site-specific integration, mutation, inversion and deletion (Dunaway et al. 1997 Molecular & Cellular Biology 17:182-189; Dymecki 1996 PNAS 93:6191-6196; Kilby et al. 1995 Plant Journal 8:637-652; Kilby et al. 1993 Trends in Genetics 9:413-421, Ow and Medberry 1995 Crit. Rev. Plant Sci. 14:239-261; Pósfai et al. 1994 Nucleic Acids Research 22:2392-2398; Senacoff et al. 1988 Journal of Molecular Biology 201:405-421; Stark et al. 1992 Trends in Genetics 8:432-439, Walters et al. 1999 Molecular & Cellular Biology 19:3714-3726).

Nevertheless, there are several significant limitations to the current technology for in planta genetic recombination as mediated by these recombinase enzymes. For example, one effective method of genetic transformation in many plants utilizes T-DNA transfer from Agrobacterium into the plant genome (Bechtold and Pelletier 1998 Methods in Molecular Biology 82:259-66; Breyne et al. 1992 Molecular & General Genetics 235:389-396; Zupan and Zambryski 1995 Plant Physiology 107:1041-1047), but many eukaryotic promoters function in Agrobacterium, and if both a recombinase and its excision target are present, excision of the transgene nucleic acid will occur in the Agrobacterium. Most strategies, therefore, require at least two separate and distinct transformation events, one delivering the target sites in one nucleic acid molecule to one plant and a second to deliver the recombinase gene in a second nucleic acid molecule to a second plant. Recombination at the target site occurs in F₁ progeny from the cross-pollination of two homozygous plants. This process generates significant time delay and extra expense in recalcitrant species such as soybean and maize. In addition, large scale production of field crops requires multiplication of seed. If the transgenes are not linked genetically, then considerable effort and expense is required to make the plants homozygous for both transgenes before seed multiplication.

Self-excising nucleic acids eliminate the need for a second transformation and additional breeding. Chemically controlled self-excising nucleic acids have been demonstrated using the R recombinase (Ebinuma and Komamine 2000, U.S. Pat. No. 5,965,791); however, both of these applications involve chemically induced excision of selectable marker genes with a recombinase while retaining the agronomically important (trait) genes. Thus, there exists a need for self-excising recombinase constructs that effectively remove all transgenic nucleic acid sequences that encode a gene product from the transgenic plant, and thereby restore the original genetic configuration of the genome.

### SUMMARY OF THE INVENTION

The present invention describes compositions and methods for producing a transgenic plant wherein an incorporated trait and other linked transgenic polynucleotides can be removed to restore the original genetic configuration of the plant's genome. In particular, the present invention provides an isolated excisable polynucleotide comprising a desired trait polynucleotide and a recombinase polynucleotide operably linked to a promoter, all flanked by a pair of directly oriented recombination sites, wherein the recombinase activity is regulatable. In a preferred embodiment the recombinase is a φC31 recombinase. More preferably, the φC31 recombinase contains an intron such that the recombinase is not expressed in bacteria such as Agrobacteria, but the recombinase is expressed in eukaryotes such as plants. In another embodiment, expression in bacteria is limited through the use of a promoter that is active in eukaryotes such as plants, but inactive in bacteria such as Agrobacteria.

The present invention allows for removal of an incorporated trait and other associated transgenic polynucleotide sequences in a transgenic plant. Such removal is desirable as it can reduce or eliminate the presence of unwanted nucleic acids in agricultural food products. The compositions and methods of the present invention also provide a means to prevent the escape of certain transgenic traits into the environment (i.e., other plants). Elimination or reduction of the escape of the transgenic traits is achieved through the use of novel self-excising recombinase cassettes containing specific regulatory sequences, such as developmentally regulated promoters, environmentally regulated promoters or a combination of developmentally and environmentally regulated constructs.

Developmentally regulated promoters used in the present invention include, but are not limited to, seed-preferred, leaf-preferred, root-preferred, pollen-preferred, egg-preferred promoter, germination-preferred, meristem-preferred, tuber-preferred, ovule-preferred and anther-preferred promoters. Preferred promoters are seed-preferred, germination-preferred and pollen-preferred promoters. In these preferred embodiments, the escape of a transgenic trait can be prevented or reduced by activating expression of the recombinase and excision of the trait specifically in propagative tissues.

In other embodiments of the present invention, recombinase activity is environmentally regulated. Preferred environmental factors and conditions, include but are not limited to, heat-shock, pathogen attack, anaerobic conditions, elevated temperature, decreased temperature, the presence of light and chemicals. In further preferred embodiments, the recombinase activity is repressible such that the desired trait is constitutively excised unless such excision is actively repressed. For example, included in the present invention is an excisable polynucleotide comprising a desired trait polynucleotide and a recombinase polynucleotide operably linked to a promoter, all flanked by a pair of directly oriented recombination sites, wherein the promoter is repressed by a chemical. The excisable polynucleotide may contain a transactivator system through which the chemical acts. Alternatively, repression of the recombinase activity can be achieved through the use of a recombinase/nuclear receptor chemical ligand domain fusion protein that sequester the recombinase and thereby prevent its translation.

Also included in the present invention are plant cells, plants, plant parts, plant seeds and trees comprising the excisable polynucleotides described herein. In one embodiment the plant seeds contain a chemical coating, wherein the chemical represses expression of the recombinase polynucleotide or represses the activity of a recombinase polypeptide encoded by the recombinase polynucleotide. The present invention also includes an isolated φC31 recombinase polynucleotide comprising an intron.

The above-described compositions can be used in various methods. In particular, the present invention includes methods of producing a transgenic plant containing an isolated excisable polynucleotide comprising the steps of 1) introducing into a plant cell the isolated excisable polynucleotide, wherein the excisable polynucleotide comprises a desired trait polynucleotide and a recombinase polynucleotide operably linked to a promoter, all flanked by a pair of recombination sites in direct orientation, wherein the recombinase polynucleotide is regulatable; and 2) generating from the plant cell the transgenic plant. The invention also includes methods of maintaining an excisable transgenic trait in a plant, comprising the steps of 1) providing a plant comprising an excisable polynucleotide, wherein the excisable polynucleotide comprises a desired trait polynucleotide and a recombinase polynucleotide operably linked to a promoter, all flanked by a pair of recombination sites in direct orientation; and 2) exposing the plant to a condition or factor that represses activity of the recombinase. Finally, the invention provides novel methods of gene stacking using the compositions described herein.

### BRIEF DESCRIPTION OF THE DRAWINGS

FIGS. 1(A-C) show schematic diagrams of T-DNAs containing self excising φC31 recombinase cassettes transcriptionally regulated by the tetracycline repressed transactivator tTA. FIG. 1D shows the T-DNA footprint that remains following excision of any of the cassettes in FIGS. 1(A-C). For all three constructs, after an excision event, only the T-DNA Footprint (<300 bp) will remain within the transgenic plant. The constituents of the cassettes are as follows: LB, left T-DNA border; pNOS, nopaline synthase promoter; codA:GmR, translational fusion of the aacCI and codA genes conferring resistance to gentamycin and sensitivity to 4-flourocytosine; pOCS, octopine synthase promoter; tTA, tetracycline repressed transactivator gene; pTOP10, Top10 promoter regulated by tTA; attB/attP, target sites for φC31 integrase; attL, recombinant product, non-target site; φC31int^{INT}, φC31 integrase gene with PIV2 intron; pSUPER, Super promoter; erGFP7^{INT}, gene for an ER localized smRS-GFP with PIV2 intron; RB, right T-DNA border; AHAS, herbicide resistance gene.

FIG. 2A shows a schematic diagram of a T-DNA containing self excising φC31 recombinase cassette translationally fused to a ligand binding domain (LBD) of a nuclear receptor protein. FIG. 2B shows the T-DNA footprint that remains following excision of the cassette. The constituents of the cassettes are as follows: LB, left T-DNA border; attB/attP, target sites for φC31 integrase; pNOS, nopaline synthase promoter; AHAS, herbicide resistance gene; attL, recombinant product, non-target site; φC31int^{INT}:LBD, φC31 integrase reading frame with PIV2 intron translationally fused to a nuclear receptor ligand binding domain; pSUPER, Super promoter; erGFP7^{INT}, gene for an ER localized smRS-GFP with PIV2 intron; RB, right T-DNA border.

FIG. 3 is a schematic diagram of a screen for reverse activity mutations in metLBD. The bottom panel describes the phenotype based upon the conditions of the screen for mutant and wild type metLBD. The URA⁺ phenotype can be screen for by the survival on minimal medium, and the URA⁻ phenotype can be screen for on complex medium supplemented with 5-fluorotic acid (5FOA). Since either phenotype can be selected, a simple survival screen can be conducted. Abbreviations used are as follows: pURA-metTA, URA3 promoter controlling the metTA gene; pTP-URA, the metTA's target promoter driving expression of the URA3 gene; JHA, juvenile hormone analog; URA⁺, survives without uracil on minimal medium; URA⁻, survives on medium supplemented with 5-fluorotic acid and uracil.

FIGS. 4(A-B) show schematic diagrams of T-DNAs containing self excising φC31 recombinase cassettes either transcriptionally regulated by the tetracycline repressed transactivator (FIG. 4A) or regulated by subcellular localization via an LBD (FIG. 4B). FIG. 4C shows a schematic diagram of a separate T-DNA containing an excisable trait gene (erGFP). FIG. 4D shows the T-DNA footprint that remains following excision of any of the cassettes shown in FIGS. 4(A-C). The constituents of the cassettes are as follows: LB, left T-DNA border; pNOS, nopaline synthase promoter; codA:GmR, translational fusion of the aacCI and codA genes conferring resistance to gentamycin and sensitivity to 4-flourocytosine; pOCS, octopine synthase promoter; tTA, tetracycline repressed transactivator gene; pTOP10, Top10 promoter regulated by tTA; attB/attP, target sites for φC31 integrase; attL, recombinant product, non-target site; φC31int^{INT}, φC31 integrase gene with PIV2 intron; φC31int^{INT}:LBD, φC31 integrase reading frame with PIV2 intron translationally fused to a nuclear receptor ligand binding domain; pSUPER, Super promoter; erGFP7^{INT}, gene for an ER localized smRS-GFP with PIV2 intron; RB, right T-DNA border; AHAS, herbicide resistance gene.

FIG. 5A shows a schematic diagram of T-DNAs containing self excising φC31 recombinase cassettes that are regulated at both the transcriptional and sub-cellular levels. To achieve this dual regulation, the reverse mutant methoprene repressed transactivator metTA is used as well as a φC31 recombinase/nuclear receptor ligand binding domain fusion polynucleotide. FIG. 5B shows the T-DNA footprint that remains following excision of the cassette. The constituents of the cassettes are as follows: LB, left T-DNA border; pNOS, nopaline synthase promoter; nptII, gene conferring resistance to kanamycin; pA9, A9 tapetum specific promoter isolated from Pinus, Arabidopsis or Brassica; metTA, methoprene repressed transactivator gene; pTPmet, promoter regulated by metTA; attB/attP, target sites for φC31 integrase; attL, recombinant product, non-target site; φC31int^{INT}, φC31 integrase gene with PIV2 intron; φC31int^{INT}:LBD, φC31 integrase reading frame with PIV2 intron translationally fused to a reverse mutant nuclear receptor ligand binding domain of the Drosophila met gene (see Example 8); pSUPER, Super promoter; erGFP7^{INT}, gene for an ER localized smRS-GFP with PIV2 intron; RB, right T-DNA border.

FIG. 6 shows the nucleotide sequence of a φC31 recombinase containing an intron (φC31int^{INT}; SEQ ID NO:9).

FIG. 7 shows the nucleotide sequence of a φC31 recombinase containing an intron (φC31int*^{INT}; SEQ ID NO: 10).

FIG. 8 shows the nucleotide sequence of construct pBPS EW051 (SEQ ID NO:11).

FIG. 9 shows the nucleotide sequence of the Arabidopsis thaliana GA4H promoter region.

### DETAILED DESCRIPTION

The present invention describes compositions and methods for producing a transgenic plant wherein an incorporated trait and other linked transgenic polynucleotides can be removed to restore the original genetic configuration of the plant's genome. Such removal of an incorporated trait and other associated transgenic polynucleotide sequences is desirable as it reduces or eliminates the presence of unwanted nucleic acids in agricultural food products. Additionally, the compositions and methods of the present invention provide a means to prevent the escape of certain transgenic traits into the environment (i.e., other plants). Elimination or reduction of the escape of the transgenic traits is achieved through the use of novel self-excising recombinase cassettes containing specific regulatory sequences such as chemically or environmentally regulated promoters.

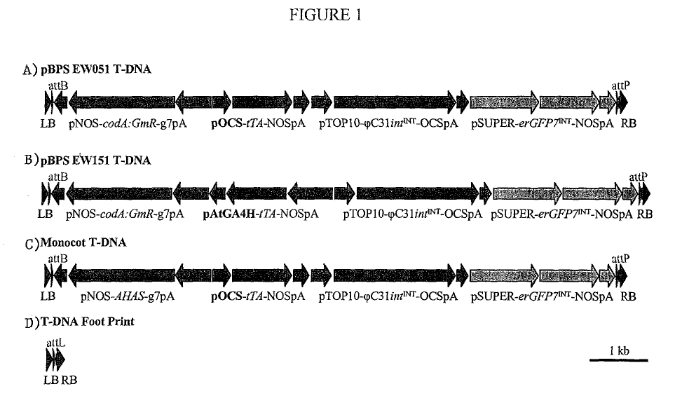

More specifically, the present invention provides an isolated excisable polynucleotide that contains a recombinase polynucleotide and a desired trait polynucleotide that are flanked by a pair of directly oriented recombination sites. Upon introduction of the polynucleotide into a eukaryote, and more preferably a plant, expression of the recombinase polynucleotide can be controlled by various regulatory polynucleotide sequences as described in more detail below. It is preferred that the recombinase polynucleotide is repressible. As used herein, a “repressible” polynucleotide, is a constitutively expressed polynucleotide, which expression can be repressed by exposure of the excisable polynucleotide to a composition such as a chemical composition. It is to be understood that repression of expression does not require complete absence of expression, but only requires a reduction in expression. Once the recombinase is expressed, it catalyzes recombination between the two directly oriented recombination sites. Recombination results in excision of the excisable polynucleotide containing both the desired trait and the recombinase and only a very small foot print remains in the eukaryotic genome. The foot print commonly only contains the sequences used for the transfer of the excisable polynucleotide into the eukaryote, such as the left and right border sequences from Agrobacterium tumefaciens, and a modified recombination site (See FIG. 1D).

There are many desired traits that can be introduced into a eukaryotic organism, and more particularly, a plant. The desired trait can be, for example, increased production of an oil or fatty acid, increased resistance to an environmental or other stress condition, increased nutritional content as through an increase in a particular vitamin, amino acid, or the like, or more simply, increased production of a polypeptide encoded by the desired trait gene. It will be understood by those of skill in the art that the “desired trait” is not limited by the present invention and encompasses any gene that can be expressed in a eukaryotic cell.

As used herein, the term “recombinase polynucleotide” refers to a polynucleotide that encodes a recombinase polypeptide that catalyzes the restriction, excision, inversion, insertion, or translocation of DNA. More preferably, the recombinase polynucleotide catalyzes recombination between two complementary recombination sites. The present invention provides excisable polynucleotides that contain both a recombinase polynucleotide and a desired trait polynucleotide flanked by two directly oriented complementary recombination sites such that expression of the recombinase polynucleotide results in excision of the recombinase polynucleotide and the desired trait polynucleotide. The term “excisable polynucleotide” therefore refers to a polynucleotide flanked by two directly oriented complementary recombination sites.

It is to be understood that the term “recombinase” includes both irreversible and reversible recombinases, transposes and integrases. In a preferred embodiment, the recombinase comprises an intron that prevents expression of the recombinase in Agrobacterium, but does not prevent expression of the recombinase in eukaryotes. The term “recombination site” refers to a nucleotide sequence that is recognized by a recombinase and that can serve as a substrate for a recombination event. The term “recombination site” also encompasses the use of “pseudo-recombination sites.” Pseudo-recombination sites are polynucleotide sequences that occur naturally in eukaryotic chromosomes and can serve as a substrate for a recombinase. Pseudo-recombination sites are described in, for example, PCT Application No. PCT/US99/18987 (WO 00/11155).

More generally, the terms “nucleic acid” and “polynucleotide” refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR and in vitro or in vivo transcription.

An “isolated” nucleic acid molecule is one that is substantially separated from other nucleic acid molecules that are present in the natural source of the nucleic acid (i.e., sequences encoding other polypeptides). Preferably, an “isolated” nucleic acid is free of some of the sequences that naturally flank the nucleic acid (i.e., sequences located at the 5′ and 3′ ends of the nucleic acid) in its naturally occurring replicon. For example, a cloned nucleic acid is considered isolated. In various embodiments, the isolated PKSRP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a plant cell). A nucleic acid is also considered isolated if it has been altered by human intervention, or placed in a locus or location that is not its natural site, or if it is introduced into a cell by agroinfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparations or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

As also used herein, an “irreversible recombinase” is defined herein as a recombinase that can catalyze recombination between two complementary recombination sites, but cannot catalyze recombination between the hybrid sites that are formed by this recombination without the assistance of an additional factor. Irreversible recombinase polypeptides, and nucleic acids that encode the recombinase polypeptides, are described in the art and can be obtained using routine methods. For example, a vector that includes a nucleic acid fragment that encodes the φC31 integrase is described in U.S. Pat. No. 5,190,871 and is available from the Northern Regional Research Laboratories, Peoria, Ill. 61604 under the accession number B-18477. Examples of other irreversible recombinases include, a coliphage P4 recombinase (Ow & Ausubel, 1983 J. Bacteriol. 155: 704-713), a coliphage lambda integrase (Lorbach et al., 2000 J. Mol. Biol., 296:1175-81), a Listeria A118 phage recombinase (Loessner et al., 2000 Mol. Micro. 35: 324-340), and an actinophage R4 Sre recombinase (Matsuura et al., 1996 J. Bacteriol. 178: 3374-3376).

One example of an irreversible recombinase and its corresponding recombination sites is the φC31 integrase and the attB and attP sites. The φC31 integrase catalyzes only the attB x attP reaction in the absence of an additional factor not found in eukaryotic cells. The recombinase cannot mediate recombination between the attL and attR hybrid recombination sites that are formed upon recombination between attB and attP. Because recombinases such as the φC31 integrase cannot alone catalyze the reverse reaction, the φC31 attB x attP recombination is stable.

It is to be understood that recombination sites generally have an orientation, or in other words, they are not palindromes. The recombination sites typically include left and right arms separated by a core or spacer region. Thus, an attB recombination site consists of BOB′, where B and B′ are the left and right arms, respectively, and O is the spacer region. Similarly, attP is POP′, where P and P′ are the arms and O is again the spacer region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP′ and POB′, respectively. The orientation of the recombination sites in relation to each other can determine which recombination event takes place. The recombination sites may be in two different orientations: directly oriented (same direction) or oppositely oriented. When the recombination sites are present on a single nucleic acid molecule and are directly oriented with respect to each other, then the recombination event catalyzed by the recombinase is typically an excision of the intervening nucleic acid. When the recombination sites are oppositely oriented, then any intervening sequence is typically inverted. In the present invention, it is preferred that the two complementary recombination sites that flank the desired trait polynucleotide and the recombinase polynucleotide are directly oriented. It is to be understood, however, that the term “flanked by” does not require that each polynucleotide sequence be located directly adjacent to a recombination site. For example, three polynucleotide sequences (A, B and C) may be flanked by recombination sites even though polynucleotide sequence B is not directly adjacent to these sites. Accordingly, the term “flanked by” is equivalent to being “in between” the recombination sites.

Similar to irreversible recombinases, reversible recombinases catalyze recombination between two complementary recombination sites. The recombinase and recombination sites are termed “reversible” because the product-sites generated by recombination are themselves substrates for subsequent recombination. Suitable reversible recombinase systems are well known to those of skill in the art and include, for example, the Cre-lox system. In the Cre-lox system, the recombination sites are referred to as “lox sites” and the recombinase is referred to as “Cre”. When lox sites are directly oriented (i.e., in the same direction), then Cre catalyzes a deletion of the intervening polynucleotide sequence. When lox sites are in the opposite orientation, the Cre recombinase catalyzes an inversion of the intervening polynucleotide sequence. This system functions in various host cells, including Saccharomyces cerevisiae (Sauer, B., 1987 Mol Cell Biol. 7:2087-2096); mammalian cells (Sauer et al., 1988 Proc. Nat'l. Acad. Sci. USA 85:5166-5170; Sauer et al., 1989 Nucleic Acids Res. 17:147-161); and plants such as tobacco (Dale, et al., 1990 Gene 91:79-85) and Arabidopsis (Osborne et al., 1995 Plant J. 7(4):687-701). Use of the Cre-lox recombinase system in plants is also described in U.S. Pat. No. 5,527,695 and PCT application No. WO 93/01283. Several different lox sites are known, including lox511 (Hoess R. et al., 1986 Nucleic Acids Res. 14:2287-2300), lox66, lox71, lox76, lox75, lox43, lox44 (Albert H. et al., 1995 Plant J. 7(4): 649-659).

Several other recombination systems are also suitable for use in the present invention. These include, for example, the FLP/FRT system of yeast (Lyznik, L. A. et al., 1996 Nucleic Acids Res. 24(19):3784-9), the Gin recombinase of phage Mu (Crisona, N.J. et al., 1994 J. Mol. Biol. 243(3):437-57), the Pin recombinase of E. coli (see, e.g., Kutsukake K, et. al, 1985 Gene 34(2-3):343-50), the PinB, PinD and PinF from Shigella (Tominaga A et al., 1991 J. Bacteriol. 173(13):4079-87), the R/RS system of the pSR1 plasmid (Araki, H. et al., 1992 J. Mol. Biol. 225(1):25-37) and the cin, hin and β recombinases. Other recombination systems relevant to this invention described herein are those from Kluyveromyces species, phages, and integrating viruses. Thus, recombinase systems are available from a large and increasing number of sources. In one embodiment of the present invention, the reversible recombinase is Cre and the recombination sites are lox sites. In a preferred embodiment, the lox sites are directly oriented. Also included within the present invention are integrase systems, such as the SSV1-encoded integrase and its corresponding recombination sites and transposase recombination systems.

In further embodiments of the present invention the isolated excisable polynucleotide contains one or more promoters. Preferably, a first promoter is operably linked to the recombinase polynucleotide and a second promoter is operably linked to the desired trait polynucleotide. “Promoter” refers to a region of DNA involved in binding the RNA polymerase to initiate transcription. A polynucleotide sequence is “operably linked” when placed into a functional relationship with another polynucleotide sequence. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, polynucleotide sequences that are operably linked are contiguous, and in the case of a signal sequence both contiguous and in reading phase. However, enhancers, for example, need not be contiguous with the coding sequences whose transcription they control. Linking is accomplished by ligation at convenient restriction sites or at adapters or linkers inserted in lieu thereof.

A promoter can be naturally associated with the recombinase polynucleotide or the desired trait polynucleotide, or it can be a heterologous promoter that is obtained from a different gene, or from a different species. Where direct expression of a gene in all tissues of a transgenic plant or other organism is desired, one can use a “constitutive” promoter, which is generally active under most environmental conditions and states of development or cell differentiation. Suitable constitutive promoters for use in plants include, for example, the cauliflower mosaic virus (CaMV) 35S transcription initiation region and region VI promoters, the 1′- or 2′- promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, the mannopine synthase promoter (MAS), various ubiquitin or polyubiquitin promoters derived from, inter alia, Arabidopsis (Sun and Callis, 1997 Plant J. 11(5):1017-1027), the mas, Mac or DoubleMac promoters (described in U.S. Pat. No. 5,106,739 and by Comai et al., 1990 Plant Mol. Biol. 15:373-381) and other transcription initiation regions from various plant genes known to those of skill in the art. Such genes include for example, ACT11 from Arabidopsis (Huang et al., 1996 Plant Mol. Biol., 33:125-139), Cat3 from Arabidopsis (GenBank No. U43147, Zhong et al., 1996 Mol. Gen. Genet., 251:196-203), the gene encoding stearoyl-acyl carrier protein desaturase from Brassica napus (GenBank No. X74782, Solocombe et al., 1994 Plant Physiol. 104:1167-1176), GPc1 from maize (GenBank No. X15596, Martinez et al., 1989 J. Mol. Biol. 208:551-565), and Gpc2 from maize (GenBank No. U45855, Manjunath et al., 1997 Plant Mol. Biol. 33:97-112).

Other useful promoters for plants also include those obtained from Ti- or Ri-plasmids, from plant cells, plant viruses or other hosts where the promoters are found to be functional in plants. Bacterial promoters that function in plants, and thus are suitable for use in the methods of the invention include the octopine synthetase promoter and the nopaline synthase promoter. Suitable endogenous plant promoters include the ribulose-1,6-biphosphate (RUBP) carboxylase small subunit (ssu) promoter, the α-conglycinin promoter, the phaseolin promoter, the ADH promoter, and heat-shock promoters.

In several preferred embodiments of the present invention, at least one constitutive promoter contained within the excisable polynucleotide sequence is capable of being regulated. As used herein, the term “regulatable promoter” refers to a promoter that directs expression of a gene where the level of expression is alterable by an environmental or developmental factor or composition such as, for example, temperature, pH, a transcription factor or a chemical. In one embodiment of the present invention, the promoter operably linked to the recombinase polynucleotide is a regulatable promoter. In another embodiment, the promoter operably linked to the recombinase polynucleotide is a constitutive promoter that can be directly or indirectly regulated by an environmental or developmental factor. In a preferred embodiment, the regulatable promoter is repressible, and more preferably, the regulatable promoter is repressed by the addition of a chemical.

Both environmentally and developmentally regulated systems, and more specifically, promoters, can be used in the present invention in order to prevent the escape of certain transgenic traits into the environment. In one embodiment, the present invention provides compositions and methods that provide activation of expression of the recombinase polynucleotide upon exposure of the plant to an environmental factor such as a chemical. In another embodiment, the compositions and methods of the present invention provide for constitutive expression of a self-excising recombinase polynucleotide in a plant until the plant is exposed to a negative environmental regulator. Accordingly, a trait can be maintained in a plant through application of the negative regulator (or an activator thereof). When and if the trait gene strays into a non-regulated plant, the recombinase is no longer repressed and the trait and recombinase excised. Environmentally regulated promoters useful in the present invention include, but are not limited to, those promoters activated or repressed by heatshock, pathogen attack, anaerobic conditions, ethylene or other chemicals, elevated temperature, decreased temperature or the presence of light. It is to be understood that the term “chemical” is not limited by the present invention and includes all natural and synthetic chemical compositions including water. Preferred chemicals are those that are non-toxic to eukaryotes, and more specifically, plants. Further preferred chemicals are agricultural chemicals (those safe to use on agricultural products as determined by the U.S. Environmental Protection Agency), and more preferably, those that are known to activate or repress a promoter that functions in a plant. Examples of preferred chemicals include plant growth regulators such as auxins, cytokinins, gibberellins, steroids, jasmonic acid and salicylic acid; tetracycline derivatives; methoprenes and other juvenile hormone receptor (JHR) analogs; and chemicals that inhibit insect development or growth such as ecdysone.

In a preferred embodiment of the present invention, a chemically repressible construct is contained within the excisable polynucleotide. In this embodiment, the excisable polynucleotide comprises a desired trait polynucleotide and a recombinase polynucleotide operably linked to a constitutive but chemically repressible promoter. In a further preferred embodiment, the excisable polynucleotide further comprises a transactivator polynucleotide operably linked to a constitutive promoter. FIGS. 1(A-C) show various examples of such preferred constructs. Upon introduction of these constructs into a host cell such as a plant cell, addition of a tetracycline derivative to the cell inhibits binding of tTA to the TOP10 promoter, thereby repressing expression of the φC31 recombinase gene. In these constructs, an ER localized GFP gene (erGFP) is used to model an agronomic trait gene (or desired trait polynucleotide) and the TOP10 promoter is used to model the chemically repressible promoter operably linked to the recombinase polynucleotide. The other construct constituents are as follows: LB (left DNA border), pNOS (nopaline synthase promoter), codA:GmR (translational fusion of the aacCI and codA genes conferring resistance to gentamycin and sensitivity to 4-flourocytosine), pOCS (octopine synthase promoter), tTA (tetracycline repressed transactivator gene), pTOP10 (Top10 promoter regulated by tTA), attB/attP (target sites for φC31 recombinase), attL (hybrid recombination site), φC31 int^{INT} (φC31 recombinase with PIV2 intron), pSUPER (super promoter), erGFP7^{INT} (gene for ER localized smRS-GFP with PIV2 intron), RB (right T-DNA border), AHAS (imidazolinone resistance gene). The pBPS EW051 T-DNA is designed to model a trait containment strategy with a constitutive promoter for tTA. The pBPS EW151 T-DNA is designed to model a trait containment strategy with a germination specific promoter. Notably, in FIG. 1C, the excisable polynucleotide further comprises a selectable marker (the modified AHAS gene).

The use of chimeric transactivators (Weinmann et al. 1994 Plant Journal 5:559-569; Aoyama et al. 1997 Plant Journal 11:605-612; Martinez et al. 1999 Plant Journal 19:97-106) offers several advantages over conventional plant regulatory systems for this regulatory control. Primarily, since their target sites are not recognized by endogenous plant transcription factors, they offer the highest level of control over gene expression (i.e. offer the least background in the “off” state). A specific example described herein is the tetracycline repressed gene regulation system (Böhner and Gatz 2001 Molecular & General Genetics 264(6):860-870, Böhner et al. 1999 Plant Journal 19:87-91, Love et al. 2000 Plant Journal 21:579-58, Weinmann et al. 1994 Plant Journal 5:559-569). This promoter, together with the φC31int^{INT} recombinase gene, ensures that premature excision during plant transformation does not occur.

Other promoter systems encompassed by the present invention that are positively regulated include, but are not limited to, the derivative of the tetracycline-induced ‘Triple-Op’ (Gatz and Quail 1988 Proc. Natl. Acad. Sci. USA 85:1394-1397), glucocorticoid-inducible ‘GAL4-UAS’ promoter (Aoyama and Chua 1997 Plant Journal 11:605-612), the ecdysone-inducible ‘GRHEcR’ promoter (Martinez et al. 1999 Plant Journal 19:97-106), or other steroid/insecticide receptor domain chimeras, especially including those derived from ecdysone or juvenile hormone III receptors (e.g. Ultraspiracle Protein [USP], Met). To date, the tTA and TGV (Böhner et al.1999 Plant Journal 19:87-91) systems are chemically repressed systems based upon the tetracycline repressor DNA binding domain; the Top10 (Weinmann et al. 1994 Plant Journal 5:559-569), TAX (Böhner et al. 1999 Plant Journal 19:87-91), TF and TFM promoters use these two chemically repressed systems.

Since any of the chemically inducible systems can generate a repressible system by altering minor components (e.g. switch ‘GAL4-UAS’ system to repressible by replacing the activation domain in GVG with a repressor domain and the minimal promoter with a GAL4 linked biologically active promoter), the term “chemical gene switch” (CGS) is used herein to refer to a chemically regulated promoter controlled by a chimeric transactivator or a repressor. Therefore, the present invention includes an excisable polynucleotide comprising a desired trait polynucleotide, a recombinase polynucleotide operably linked to a chemical gene switch.

In the previous examples, a gene switch (e.g. tTA/pTOP) system supplied chemical regulation of the recombinase polynucleotide. However, chemical regulation can also be operationally linked to the recombinase polynucleotide by translation fusion or interaction domains with the ligand receptor domain of a nuclear receptor. Thus, control of the recombinase polynucleotide can be achieved not only at the transcriptional level, but also at the subcellular localization level by operationally linking the recombinase protein with a nuclear receptor domain to control activity by subcellular localization as described by U.S. Pat. No. 6,040,430, or logical extensions thereof. Since recombinase polynucleotide localized to the cytosol cannot bind to nuclear localized DNA because of the physical separation, recombinase activity becomes regulated by the chemical ligand.

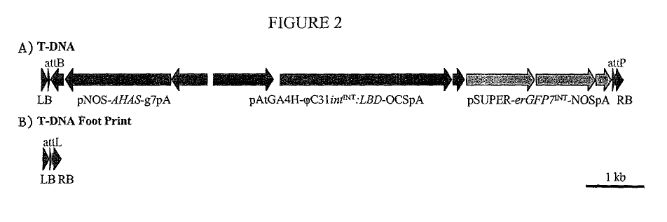

Therefore, chemical repression can be achieved at the transcriptional level, the subcellular localization level, or by both in concert (e.g. replace φC31int^{INT} genes in FIG. 1 with φC31int^{INT}:LBD genes). FIG. 2 shows one example of chemical repression achieved at the subcellular localization level. In FIG. 2, the φC31int^{INT}:LBD gene is a φC31 recombinase reading frame with a PIV2 intron translationally fused to a nuclear receptor ligand binding domain. Descriptions of the other constituents can be found above in relation to FIG. 1.

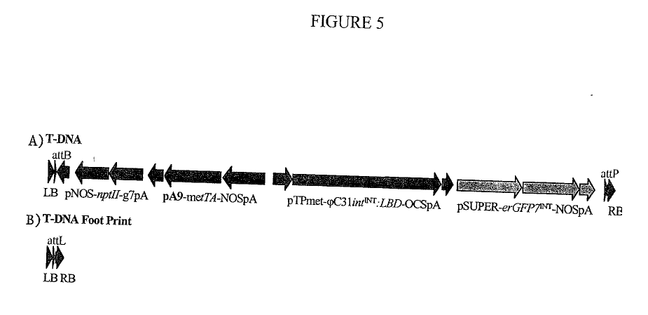

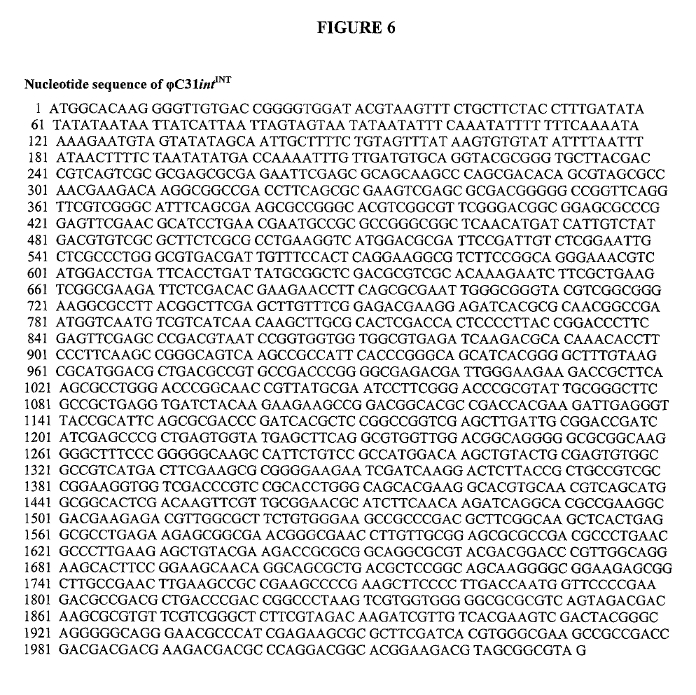

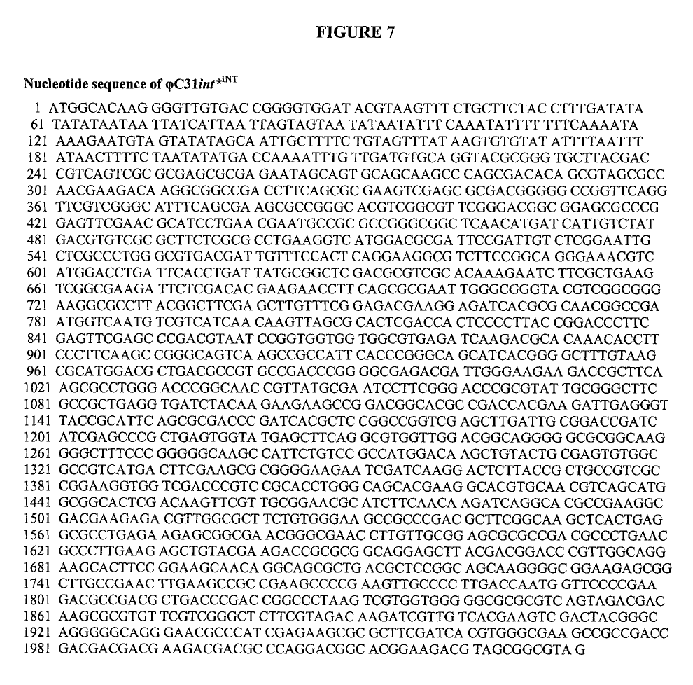

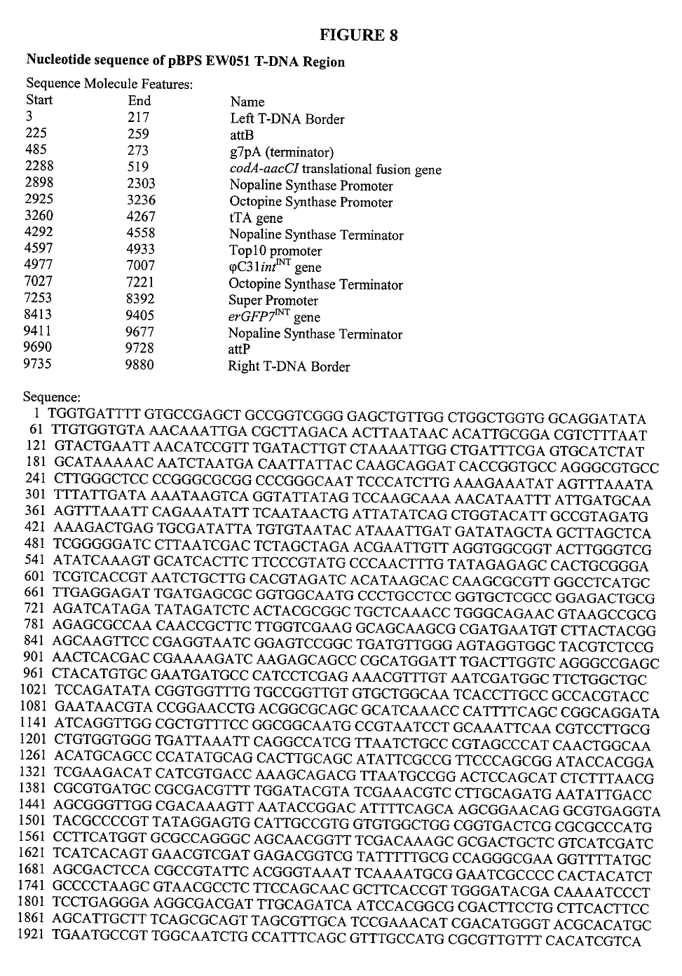

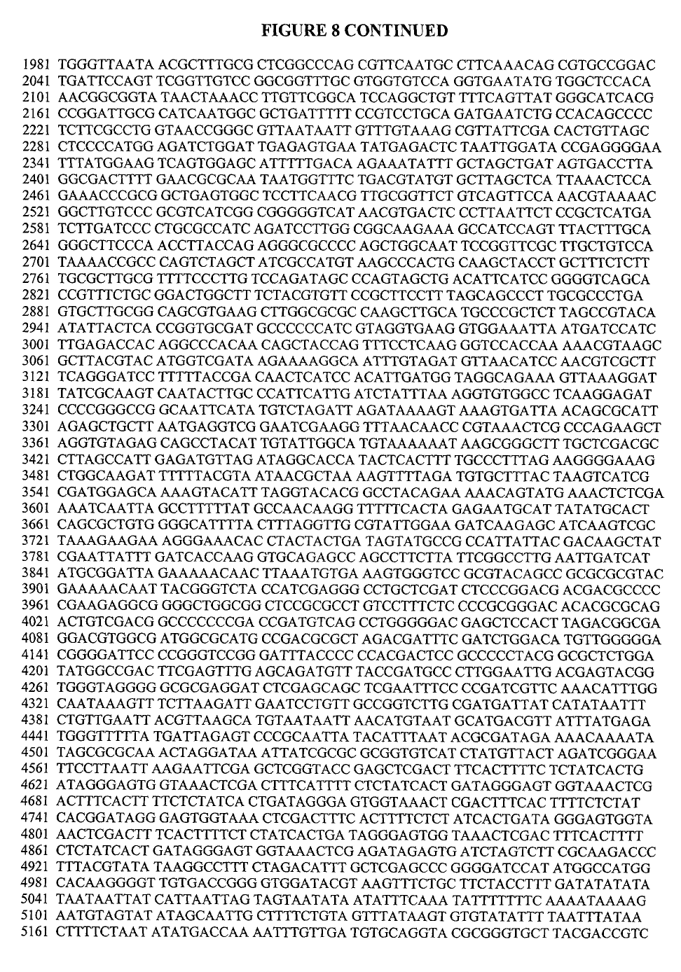

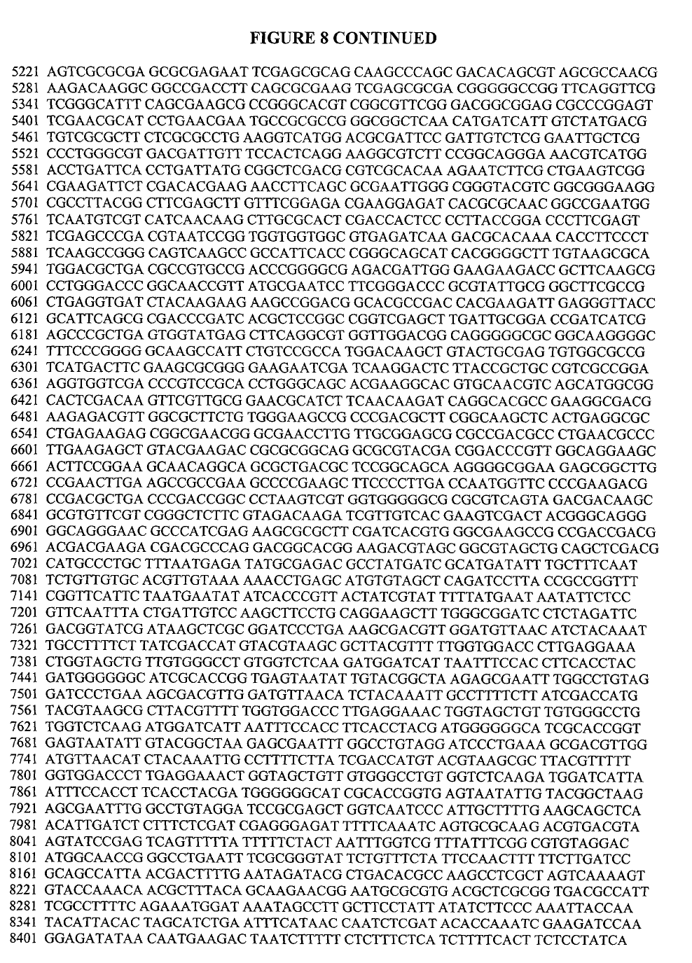

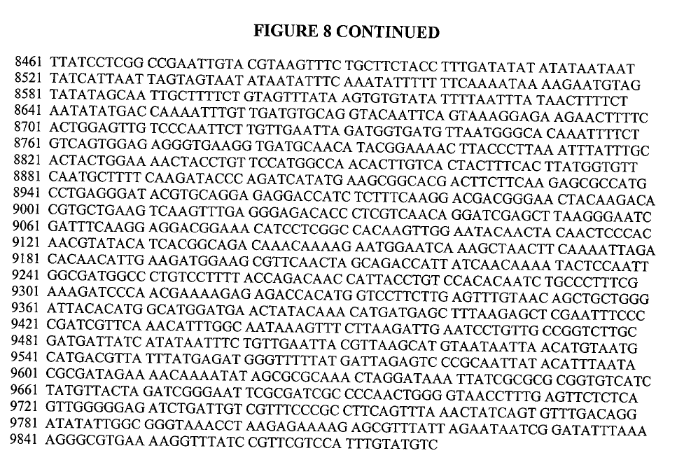

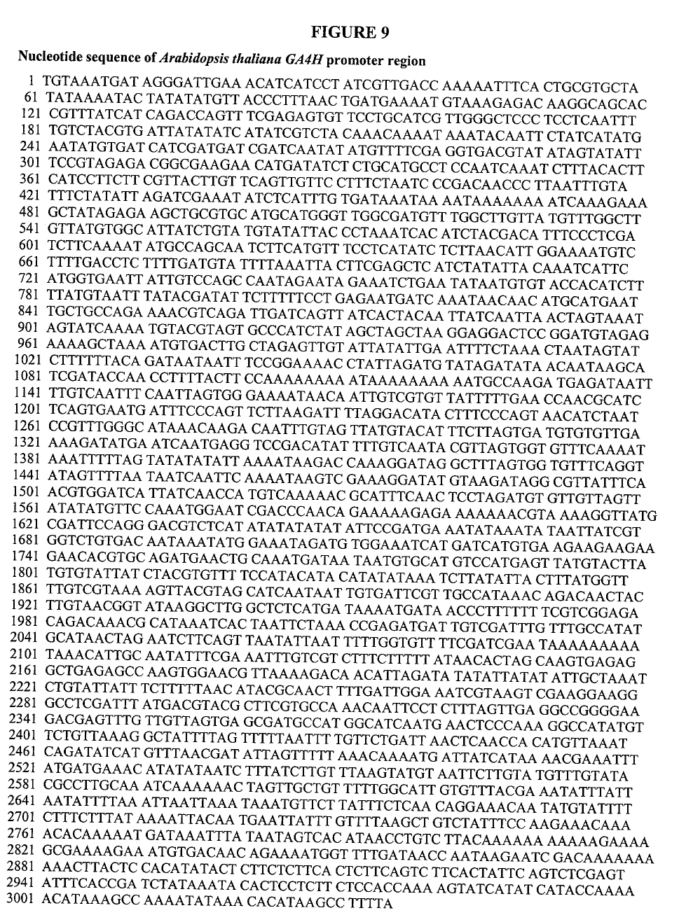

Accordingly, the present invention includes an isolated excisable polynucleotide comprising a desired trait polynucleotide and a recombinase polynucleotide operably linked to a ligand binding domain of a nuclear receptor protein. While only the subcellular localization aspect is described in FIG. 2, the present invention encompasses both the subcellular localization and the combination of the transcriptional and subcellular level control of the recombinase polynucleotide (i.e., an isolated excisable polynucleotide comprising a desired trait polynucleotide, a recombinase polynucleotide operably linked to a ligand binding domain of a nuclear receptor protein and a regulatable promoter. (See FIG. 5 and Example 10). The terms “nuclear receptor” and “steroid receptor”, as used herein, refers to a molecule, preferably a protein molecule, whose subcellular localization (e.g. nuclear v. cytosol) is modulated by binding of a ligand. The terms “nuclear receptor” and “steroid receptor” include the ecdysone, USP, Drosophila met, glucocorticoid, testosterone and estrogen receptors or their derivatives and other receptors described in U.S. Pat. No. 6,040,430 and Laudet et al. (1992 EMBO Journal 11(3):1003-1013). The term “derivatives” refers to receptors that remain regulatable by a ligand obtained by deletion, mutation or substitution, including codon optimization or alterations. Most receptor domains of this class are localized to the cytoplasm in the absence of ligand; however, in the presence of bound ligand, translocation into the nucleus allows the transactivator to bind DNA and trigger transcription or repression (Laudet et al. 1992 EMBO Journal 11(3):1003-1013). Similarly, steroid type receptor domains translationally linked to recombinase proteins impose chemical regulation upon the linked recombinase activity (Nichols et al. 1997 Molecular Endocrinology 11(7):950-961; U.S. Pat. No. 6,040,430).

In another embodiment of the present invention, developmentally regulated systems are used to affect excision of the desired trait gene from the polynucleotides of specific tissues at specific times. These developmentally regulated systems can be used to completely or partially reduce the inheritance of the excisable polynucleotides in plants if the developmental regulation is related to the formation of male or female gametes.

The developmentally regulated systems used in the present invention comprise developmentally regulated promoters. Accordingly, the present invention includes an isolated excisable polynucleotide comprising a desired trait polynucleotide, a recombinase polynucleotide operably linked to a developmentally regulated promoter. Developmentally regulated promoters include promoters that initiate transcription only in certain tissues, such as leaves, roots, fruit, seeds, or flowers. In one embodiment of the present invention, the developmentally regulated promoter is a seed-preferred promoter. In another embodiment, the developmentally regulated promoter is a pollen-preferred promoter. The use of a pollen-preferred promoter linked to the recombinase gene can be used to prevent the transmission of the excisable polynucleotide through pollen. More specifically, the excisable polynucleotide will be contained within the female and thereby reduce outcrossing to wild relatives in crop species that are naturally cross-pollinating including maize, grasses (including turf and forage species) and rapeseed.

The pollen promoter is simply an example of a promoter that is regulated in a specific developmental manner. By the careful choice of promoter, the recombinase transgene can be activated in specific tissue and at a specific stage of development. Chimeric plants can therefore be created through the use of promoters that are active in vegetative tissues. Other examples of tissue preferred and organ preferred promoters include, but are not limited to, ovule-preferred, male tissue-preferred, integument-preferred, tuber-preferred, stalk-preferred, pericarp-preferred, stigma-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed-preferred promoters can be embryo-preferred, endosperm-preferred and seed coat-preferred. See Thompson et al. 1989 BioEssays 10:108. Examples of seed-preferred promoters include, but are not limited to cellulose synthase (celA), Cim1, gamma-zein, globulin-1, maize 19 kD zein (cZ19B1) and the like.

Other suitable tissue-preferred or organ-preferred promoters include the napin-gene promoter from rapeseed (U.S. Pat. No. 5,608,152), the USP-promoter from Vicia faba (Baeumlein et al. 1991 Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980) or the legumin B4 promoter (LeB4; Baeumlein et al. 1992 Plant Journal, 2(2):233-9) as well as promoters conferring seed specific expression in monocot plants like maize, barley, wheat, rye, rice, etc. Suitable promoters to note are the lpt2 or lpt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene and rye secalin gene).

Additionally, the tissue-specific E8 promoter from tomato is particularly useful for directing gene expression so that a desired gene product is located in fruits. See, e.g., Lincoln et al., 1988 Proc. Nat'l. Acad. Sci. USA 84: 2793-2797; Deikman et al., 1988 EMBO J. 7: 3315-3320; Deikman et al., 1992 Plant Physiol. 100: 2013-2017. Other suitable promoters include those from genes encoding embryonic storage proteins. Additional organ-specific, tissue-specific and/or inducible foreign promoters are also known (see, e.g., references cited in Kuhlemeier et al., 1987 Ann. Rev. Plant Physiol. 38:221), including those 1,5-ribulose bisphosphate carboxylase small subunit genes of Arabidopsis thaliana (the “ssu” promoter), which are light-inducible and active only in photosynthetic tissue, anther-specific promoters (EP 344029), and seed-specific promoters of, for example, Arabidopsis thaliana (Krebbers et al., 1988 Plant Physiol. 87:859). Exemplary green tissue-specific promoters include the maize phosphoenol pyruvate carboxylase (PEPC) promoter, small submit ribulose bis-carboxylase promoters (ssRUBISCO) and the chlorophyll a/b binding protein promoters. The promoter may also be a pith-specific promoter, such as the promoter isolated from a plant TrpA gene as described in International Publication No. W0/93/07278.

The combination of chemical and developmental control of a recombinase gene provides a novel means to remove transgenes from plants and thereby restrict their distribution in the environment, yet enable seed multiplication. Recombinase enzymes can be used to excise transgenes from transgenic plants at specific recombination sites incorporated in the transgene, and thereby prevent the transmission of the transgenes to sexual or vegetative progeny of the crop. This invention provides developmental and tissue-specific control of recombinase expression that is important for application of this technology in field crops, such as soybean or maize. Genes that are solely transcribed in seeds, leaves, tubers, roots or other specific cells and tissues are well known in the literature and their promoters can be used to provide developmental regulation of recombinase gene expression. The precise characteristics required of this developmental promoter differ depending on the trait and application. For example, if the trait is a phenotype present in only vegetative tissue, the recombinase can be controlled by a promoter that is active during flower or seed development. In this example, the transgene will be removed from most seeds. Examples of these traits include disease, insect and herbicide resistance. As a second example, if the trait is a characteristic of the seed or flower, the recombinase cannot be controlled by a promoter that is active during flower or seed development. Instead, the recombinase must be active only after seed development is completed, or in other words, during germination. Traits that are seed phenotypes include modifications of oil, starch and protein content.

Any of these promoters can be combined with a chemical gene switch to control expression and, therefore, control the timing of transgene excision from specific cells. The present invention therefore includes an isolated excisable polynucleotide comprising a comprising a desired trait polynucleotide and a recombinase polynucleotide operably linked to both a developmentally regulated promoter and a chemical gene switch. Modern production of crops, such as soybean and corn, requires multiplication of genetically uniform seeds. This is achieved by successive rounds of self and/or cross-pollination. Clearly, excision of a transgenic trait during seed multiplication provides no advantage and therefore recombinase activity must be suppressed during the seed multiplication phase. One means to achieve transient control of recombinase gene expression is by use of a chemical gene switch such that a chemical can be sprayed on the flowering plant or applied to the seed prior to planting.

Generally, a polynucleotide that is to be expressed (e.g., a recombinase polynucleotide or desired trait polynucleotide) will be present in an expression cassette, meaning that the polynucleotide is operably linked to expression control sequences, e.g., promoters and terminators that are functional in the host cell of interest. Expression cassettes for use in i E. coli include the T7, trp, or lambda promoters, a ribosome binding site and preferably a transcription termination signal. For eukaryotic cells, the control sequences typically include a promoter which optionally includes an enhancer derived from immunoglobulin genes, SV40, cytomegalovirus, etc., and a polyadenylation sequence, and may include splice donor and acceptor sequences. In yeast, convenient promoters include GAL1-10 (Johnson and Davies, 1984 Mol. Cell. Biol. 4:1440-1448) ADH2 (Russell et al., 1983 J. Biol. Chem. 258:2674-2682), PHO5 (Meyhack et al., 1982 EMBO J. 6:675-680), and MFα (Herskowitz and Oshima, 1982, in The Molecular Biology of the Yeast Saccharomyces (eds. Strathem, Jones, and Broach) Cold Spring Harbor Lab., Cold Spring Harbor, N.Y., pp. 181-209).

To facilitate selection of cells in which recombinase gene has been introduced, the excisable polynucleotide sequence can include (preferably between the recombination sites) a selectable marker. Suitable examples of negative selection markers are known to those of skill in the art. Examples of selectable markers for E. coli include: genes specifying resistance to antibiotics, i.e., ampicillin, tetracycline, kanamycin, erythromycin, or genes conferring other types of selectable enzymatic activities such as β-galactosidase, or the lactose operon. Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg, 1981 Proc. Nat'l. Acad. Sci. USA, 78: 2072; Southern & Berg, 1982 J. Mol. Appl. Genet., 1:327).

Selection markers for plant cells often confer resistance to a biocide or an antibiotic, such as, for example, kanamycin, G 418, bleomycin, hygromycin, or chloramphenicol, or herbicide resistance, such as resistance to chlorsulfuron or Basta. Selection markers also include polynucleotide sequences that confer herbicide resistance, such as the AHAS gene. Examples of suitable coding sequences for selectable markers are: the neo gene which codes for the enzyme neomycin phosphotransferase which confers resistance to the antibiotic kanamycin (Beck et al., 1982 Gene, 19:327); the hpt gene, which codes for the enzyme hygromycin phosphotransferase and confers resistance to the antibiotic hygromycin (Gritz and Davies, 1983 Gene, 25:179); and the bar gene (EP 242236) that codes for phosphinothricin acetyl transferase which confers resistance to the herbicidal compounds phosphinothricin and bialaphos.

The above-described compositions and methods can be used to integrate an excisable desired trait polynucleotide into any eukaryotic cell. Non-limiting examples of the eukaryotic cells of the present invention include cells from animals, plants, fungi, bacteria and other microorganisms. In one embodiment, the eukaryotic cell is a mammalian cell. In another embodiment, the eukaryotic cell is a plant cell. In some embodiments, the cells are part of a multicellular organism, e.g., a transgenic plant or animal. Among the plant targets of particular interest are monocots, including, for example, rice, corn, wheat, rye, barley, bananas, palms, lilies, orchids, and sedges. Dicots are also suitable targets, including, for example, tobacco, apples, potatoes, beets, carrots, willows, elms, maples, roses, buttercups, petunias, phloxes, violets, sunflowers, soybeans, oilseed rapes, alfalfas, clovers, beans, peanuts, cottons and tomatoes. Finally, gymnosperms including, pines, cedars and eucalyptus can be used according to the present invention.

The excisable polynucleotide constructs described herein can be introduced into the target cells and/or organisms by any of the several means known to those of skill in the art. For instance, the DNA constructs can be introduced into plant cells, either in culture or in the organs of a plant by a variety of conventional techniques. For example, the DNA constructs can be introduced directly to plant cells using biolistic methods, such as DNA particle bombardment, or the DNA construct can be introduced using techniques such as electroporation and microinjection of plant cell protoplasts. Particle-mediated transformation techniques (also known as “biolistics”) are described in Klein et al., 1987 Nature 327:70-73; Vasil, V. et al., 1993 Bio/Technol. 11:1553-1558; and Becker, D. et al., 1994 Plant J. 5:299-307. These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants, bacteria, fungi, algae, intact animal tissues, tissue culture cells, and animal embryos. One can employ electronic pulse delivery, which is essentially a mild electroporation format for live tissues in animals and patients (Zhao, 1995 Advanced Drug Delivery Reviews, 17:257-262).

Other transformation methods are also known to those of skill in the art. Microinjection techniques are known in the art and well described in the scientific and patent literature. The introduction of DNA constructs using polyethylene glycol (PEG) precipitation is described in Paszkowski et al., EMBO J. 3:2717 (1984). Electroporation techniques are described in Fromm et al., Proc. Natl. Acad. Sci. USA 82:5824 (1985). PEG-mediated transformation and electroporation of plant protoplasts are also discussed in Lazzeri, P., Methods Mol. Biol. 49:95-106 (1995). Methods are known for introduction and expression of heterologous genes in both monocot and dicot plants. See, e.g., U.S. Pat. Nos. 5,633,446, 5,317,096, 5,689,052, 5,159,135, and 5,679,558; Weising et al., 1988 Ann. Rev. Genet. 22:421-477. Transformation of monocots in particular can be achieved using various techniques including electroporation (e.g., Shimamoto et al., 1992 Nature 338:274-276; biolistics (e.g., European Patent Application 270,356); and Agrobacterium (e.g., Bytebier et al., 1987 Proc. Nat'l Acad. Sci. USA 84:5345-5349).

For transformation of plants, DNA constructs may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-meditated transformation techniques are well described in the scientific literature. See, for example, Horsch et al., 1984 Science 233:496-498; Fraley et al., Proc. Natl. Acad. Sci. USA 80:4803 (1983); Hooykaas, Plant Mol. Biol., 13:327-336 (1989); Bechtold et al., Comptes Rendus De L Academie Des Sciences Serie Iii-Sciences De La Vie-Life Sciences 316:1194-1199 (1993); and Valvekens et al., 1988 Proc. Natl. Acad. Sci. USA 85:5536-5540. For a review of gene transfer methods for plant and cell cultures, see, Fisk et al., Scientia Horticulturae 55:5-36 (1993) and Potrykus, CIBA Found. Symp. 154:198 (1990). When Agrobacterium tumefaciens-meditated transformation techniques are used, the polynucleotide introduced into the plant may be referred to herein as T-DNA. This T-DNA comprises an excisable polynucleotide of the present invention flanked by a left and right T-DNA border (also referred to herein as LB and RB).

Other methods for delivery of polynucleotide sequences into cells include, for example, liposome-based gene delivery (Debs and Zhu 1993 WO 93/24640; Mannino and Gould-Fogerite 1988 BioTechniques 6(7):682-691; Rose U.S. Pat. No. 5,279,833; Brigham 1991 WO 91/06309; and Felgner et al., 1987 Proc. Natl. Acad. Sci. USA 84:7413-7414), as well as use of viral vectors such as papillomaviral, retroviral and adeno-associated viral vectors (e.g., Berns et al., 1995 Ann. NY Acad. Sci. 772:95-104; Ali et al., 1994 Gene Ther. 1:367-384; and Haddada et al., 1995 Curr. Top. Microbiol. Immunol. 199 (Pt 3):297-306 for review; Buchscher et al., 1992 J. Virol. 66(5):2731-2739; Johann et al., 1992 J. Virol. 66(5):1635-1640; Sommerfelt et al., 1990 Virol. 176:58-59; Wilson et al., 1989 J. Virol. 63:2374-2378; Miller et al., 1991 J. Virol. 65:2220-2224; Wong-Staal et al., PCT/US94/05700, and Rosenburg and Fauci (1993) in Fundamental Immunology, Third Edition Paul (ed) Raven Press, Ltd., New York and the references therein; Yu et al., Gene Therapy (1994) supra.; West et al., 1987 Virology 160:38-47; Carter et al., 1989 U.S. Pat. No. 4,797,368; Carter et al., 1993 WO 93/24641; Kotin 1994 Human Gene Therapy 5:793-801; Muzyczka 1994 J. Clin. Invst. 94:1351 and Samulski (supra) for an overview of AAV vectors; Lebkowski, U.S. Pat. No. 5,173,414; Tratschin et al., 1985 Mol. Cell. Biol. 5(11):3251-3260; Tratschin et al., 1984 Mol. Cell. Biol., 4:2072-2081; Hermonat and Muzyczka 1984 Proc. Natl. Acad. Sci. USA 81:6466-6470; McLaughlin et al., 1988 and Samulski et al., 1989 J. Virol. 63:03822-3828).

Methods by which one can analyze the integration pattern of the introduced excisable polynucleotide are well known to those of skill in the art. For example, one can extract DNA from the transformed cells, digest the DNA with one or more restriction enzymes, and hybridize to a labeled fragment of the polynucleotide construct. The inserted sequence can also be identified using the polymerase chain reaction (PCR). (See, e.g., Sambrook et al., Molecular Cloning—A Laboratory Manual, Cold Spring Harbor Laboratory, Cold Spring Harbor, N.Y., 1989 for descriptions of these and other suitable methods).

Transformed plant cells, derived by any of the above transformation techniques, can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, Macmillian Publishing Company, New York (1983); and in Binding, Regeneration of Plants, Plant Protoplasts, pp. 21-73, CRC Press, Boca Raton, (1985). Regeneration can also be obtained from plant callus, explants, somatic embryos (Dandekar et al., 1989 J. Tissue Cult. Meth., 12:145; McGranahan et al., 1990 Plant Cell Rep., 8:512), organs, or parts thereof. Such regeneration techniques are described generally in Klee et al., 1987 Ann. Rev. of Plant Phys. 38:467-486.

Accordingly, the present invention encompasses plant cells comprising the excisable polynucleotides described above. The present invention also includes plants, plant parts and plant seeds that contain plant cells comprising the excisable polynucleotides described above. Plant parts include, but are not limited to, stems, roots, ovules, stamens, leaves, embryos, meristematic regions, callus tissue, gametophytes, sporophytes, pollen, microspores and the like. The plant can include, but is not limited to, maize, wheat, rye, oat, triticale, rice, barley, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses and forage crops, these crop plants are also preferred target plants for a genetic engineering as one further embodiment of the present invention. Perennial grasses and forage crops include, but are not limited to, turfgrass, such as perennial ryegrass, wildryegrass, bentgrass, bluegrass, fescues, hairgrass, Koeleria cristata, Puccinellia Dis., Redtop, Timothy, trivialis, bermudagrass, buffalograss, St. Augustinegrass and zoysiagrass; wheatgrass; canarygrass; bromegrass; orchardgrass; alfalfa; salfoin; birdsfoot trefoil; alsike clover; red clover and sweet clover. Also included in the present invention are trees and tree cells comprising the excisable polynucleotide described above. The trees can include, but are not limited to, oil palm and coconut as mentioned above and gymnosperms (Pinophyta) from the following taxon: Pinals, Ginkoales, Cycadales and Gnetales.

The plant seeds of the present invention comprise the isolated excisable polynucleotides described above. In preferred embodiments, the plant seeds also comprise a chemical coating, wherein the chemical represses expression of the recombinase polynucleotide. Chemical coating methods are well known to those of skill in the art and can be found in references such as U.S. Pat. No. 6,156,699 and U.S. Pat. No. 5,290,791.

Additionally included in the present invention are methods of producing a transgenic plant containing an excisable nucleic acid comprising, introducing into a plant cell an isolated excisable polynucleotide, wherein the excisable polynucleotide comprises a recombinase polynucleotide and a desired trait polynucleotide, both flanked by a pair of recombinase target sites in direct orientation and generating from the plant the transgenic plant. The excisable polynucleotide can be any of the excisable polynucleotides described above. For example, the excisable polynucleotide can further comprise a regulatable promoter or chemical gene switch operably linked to the recombinase polynucleotides. The regulatable promoter is preferably an environmentally regulated promoter or a developmentally regulated promoter. In a more preferred embodiment, the regulatable promoter is a chemically regulated promoter.

One advantage of the plants produced by (and included within) the present invention is that it provides a means to contain uncontrolled release of trait genes into the wild. Unintentional release of transgenes into a wild population is a concern among environmental groups and regulatory agencies. Furthermore, control of trait loci is important for breeding engineered crops since unintended cross-pollination can cause costly contamination within non-genetically modified (GMO) breeding programs. Therefore, a mechanism for controlling the spread or escape of transgenes has significant utility due to both environmental and breeding concerns. The use of chemically repressed excision as described in various preferred embodiments herein results in excision being automatically stimulated in unsupervised environments.

Another advantage of the plants produced by (and included within) the present invention is the essentially complete excision of the transgenic genes from the plant's genome. This complete excision removes any fitness advantage provided by the transgenic locus. Additionally, transgenic trait removal by excision results in viable progeny with a vestigial excision footprint, and thereby does not harm, or reduce the fitness of, the population into which it has strayed.

Finally, the use of self-excising polynucleotides significantly reduces the time and cost of product development. This invention also increases the feasibility of using in planta genetic recombination technology in other applications beyond plant breeding, for example during crop production to restrict the distribution and spread of the transgenes through pollen or seeds.

Throughout this application, various publications are referenced. The disclosures of all of these publications and those references cited within those publications in their entireties are hereby incorporated by reference into this application in order to more fully describe the state of the art to which this invention pertains. It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Creation of an Intron Containing φC31 Integrase Gene

Two novel φC31 integrase genes, φC31int^{INT} and φC31int*^{INT}, containing an intron were constructed by directed, silent mutagenesis as follows. Two oligonucleotides, 5′-GATCCATATGGCCATGGCACAAGGGGTTGTGACCGGGGTGGATAC-3′ (SEQ ID NO:1) and 5′-GTACGTATCCACCCCGGTCACAACCCCTTGTGCCATGGCCATATG-3′ (SEQ ID NO:2), were used to generate a Sna BI endonuclease restriction site within the φC31int gene. To simplify future sub-cloning, the resulting gene, φC31int^{SnaBI}, was then submitted to site direct mutagenesis (Transformer Site Directed Mutagenesis Kit, Clontech) to remove the internal Hin DIII, Eco RI, and Bsi WI endonuclease restriction sites to create φC31int*. The following oligonucleotide primers were used:

5′-GAGCGCGAGAATAGCAGTGCAGCAAGCCCA-3′ (SEQ ID NO:3);

5′-GTCATCAACAAGTTAGCGCACTCGACCACT-3′ (SEQ ID NO:4);

5′-GACCGCGCGGCAGGAGCTTACGACGGACCC-3′ (SEQ ID NO:5); and

5′-CCGCCGAAGCCCCGAAGTTGCCCCTTGACC-3′ (SEQ ID NO:6).

Then, the PIV2 intron from gus^{INT} (Vancanneyt et al. 1990 Molecular & General Genetics 220:245-250) was PCR amplified using primers 5′-TTCCGCGGCCGCTACGTAAGTTTCTGCTTCTACCT-3′ (SEQ ID NO:7) and 5′-AAACAGCTGCACATCAACAAATTTTGGTCA-3′ (SEQ ID NO:8). The resulting PCR product was sub-cloned using Sna BI and Pvu II into the Sna BI sites within φC31int^{SnaBI} and φC31int* to create φC31int^{INT} (SEQ ID NO:9) and φC31int*^{INT} (SEQ ID NO:10), respectively.

For functional validation, φC31int, φC31int^{INT} and φC31int*^{INT} were subcloned into an expression cassette containing the Super promoter (pSuper [a.k.a. (ocs)₃mas], Ni et al., 1995 Plant Journal 7:661-676) and nopaline synthase terminator (Jefferson et al. 1987 EMBO Journal 6:3901-3907) resulting in plasmids pBPS LM094, pBPS LM095 and pBPS LM124, respectively. The test vector pBPS LM126 (pSuper-attP-codA-attB-gusA) was constructed using standard molecular techniques such that a Super promoter controls expression of a codA gene flanked by φC31 integrase target sites attB and attP in direct orientation (for excision) followed by a gusA gene with a nopaline synthase terminator. Active φC31 INT protein causes excision of codA in pBPS LM126 yielding an activated gusA product (pSuper-attR-gusA). Therefore, GUS activity from pBPS LM126 indirectly measures in planta φC31 INT recombinase activity.

To functionally test the new integrase genes, soybean root segments were bombarded or co-bombarded with one or two plasmids and were stained for GUS activity after 24 hours using standard protocols. GUS activity from pBPS LM126 was observed only in the presence of φC31int containing plasmids pBPS LM094, pBPS LM095 and pBPS LM124. This result confirms that the new φC31int^{INT} and φC31int*^{INT} genes function in plant cells. The GUS activity observed due to excision of codA was less than that observed from a simple pSuper-gusA plasmid. This was expected since the efficiency of excision after 24 hours was expected to be less than 100% and since there would be less time for GUS enzyme accumulation to occur relative to the pSuper-gusA control.

### Example 2

### Construction of Self-excising T-DNA for Dicotyledonous Plants

A binary vector is constructed that contains the φC31int^{INT} recombinase gene controlled by the TOP10 promoter (pTOP10), a tetracycline repressed transactivator (tTA) gene controlled by the octopine synthase promoter (pOCS) (FIG. 1, pBPS EW051). Standard sub-cloning, PCR-based sub-cloning, and oligonucleotide DNA synthesis procedures are used to construct pBPS EW051 (SEQ ID NO:11).

The self-excising cassette from vector pBPS EW051 is validated in planta using Arabidopsis thaliana as a representative dicotyledonous plant. Vacuum infiltration transformation (VIT) was performed on Arabidopsis thaliana ecotype Columbia (Col0) plants sown in screen-covered pots (Bechtold et al. 1993 C. R. Acad. Sci. Paris 316:1194-1199; Bechtold and Pelletier 1998 Methods in Molecular Biology 82:259-66; Bent et al. 1994 Science 265:1856-1860). The plants were germinated and grown with an 8 hour, 23° C. day and 16 hour 20° C. night. Cool white fluorescent lamps were used to provide light, ca. 150 μmol m⁻²s⁻¹. After adequate vegetative growth was obtained, transferring the plants to a 16 hour, 20° C. day and 8 hour, 18° C. night induced bolting. An overnight culture of Agrobacterium tumefaciens C58C1 (pMP90) transformed the appropriate binary vector plasmid was used to inoculate 0.5 L of YEB (Sambrook et al. 1989 Molecular Cloning: A Laboratory Manual. Cold Spring Harbor, N.Y.: Cold Spring Harbor Laboratory Press). The Agrobacterium were grown at 28° C. with shaking at 275 r.p.m. until the optical density at 600 nm was greater than 2.0. The bacteria were then pelleted by centrifugation (30 minutes, 3500 r.p.m.), and resuspended in 0.5 L of VIM (0.5×MS salts, 1×Gamborg's B5 vitamins, 5% sucrose, 500 mg/L MES, 44 nM benzylaminopurine, and 200 ppm Silwet L-77). Plants were vacuum-infiltrated when the bolts reached 10-15 cm tall by placing them up-side down and submerged inside a bell jar containing the resuspended Agrobacterium. A vacuum (ca. 700 mm Hg) was applied for approximately 5 minutes. Finally, the plants were drained and returned to 16 hour, 23° C. day and 8 hour, 20° C. night until seed set was complete. Cool white fluorescent lamps were used to provide light, ca. 100 μmol m⁻²s⁻¹. The resulting T₁ seed was collected and cleaned.

Some T₁ seeds are germinated on medium containing gentamycin and doxycycline, whereas others are germinated on the same medium lacking doxycycline. In the absence of doxycycline, expression of the recombinase is activated leading to excision of the transgenes in the T-DNA between the attP and attB sites. In the case of plants containing T-DNA from pBPS EW051, the plants grown in the presence of doxycycline have GFP activity, whereas those grown in its absence lack GFP activity. Similarly, seeds of the plants grown in the presence of doxycycline have tolerance to gentamycin, whereas seeds of those grown in its absence are susceptible to gentamycin. This example illustrates the principle of complete excision of all transgenes from the T-DNA in the plant's genome.

To confirm complete excision, DNA is extracted from samples of all groups of plants. DNA of those grown in the presence of doxycycline contain a positive PCR band, whereas those grown in its absence lack the same PCR band, indicating that all or part of the T-DNA has been excised from the genomic DNA of these plants. This is further confirmed by Southern hybridization. DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer. DNA samples from those grown in the presence of doxycycline contained a positive band for the T-DNA, whereas those grown in its absence lacked the band.

### Example 3

### Construction of Self-excising φC31 T-DNA for Monocotyledonous Plants

Self-excising T-DNA vectors for monocotyledonous plants also contain the recombinase gene φC31int^{INT} or φC31int*^{INT}. A tetracycline-repressed gene regulation system (Böhner and Gatz 2001 Molecular & General Genetics 264(6):860-870, Böhner et al. 1999 Plant Journal 19:87-91, Love et al. 2000 Plant Journal 21:579-588, Weinmann et al. 1994 Plant Journal 5:559-569) is used to control expression of the recombinase gene φC31int^{INT} or φC31int*^{INT}. A binary vector is constructed similar to that used in Example 2, except that the selectable marker is the modified AHAS gene for resistance to the imidazolinone herbicides (FIG. 1, Monocot T-DNA).

The self-excising φC31int cassette is validated for monocotyledonous plants in planta using perennial ryegrass (Lolium perenne) as a typical monocotyledonous plant. Plants are transformed with in the presence of doxycycline to prevent expression of the recombinase gene. Transformed plants are selected in the presence of doxycycline. For transformation of perennial ryegrass, mature caryopses are sterilized, germinated and plated on Murashige and Skoog culture medium supplemented with casein hydrolysate (500 mg/L), sucrose (20 g/L), asparagine (150 mg/L), benzyladenine (10 mg/L), dicamba (5 mg/L) and Phytagel (2 g/L) pH 5.8 (MSPR medium). Callus is incubated in the dark and transferred to fresh medium at four-week intervals. Agrobacterium tumefaciens mediated transformation of ryegrass is performed on callus using strain C58C1 (pMP90). Overnight cultures of A. tumefaciens are grown at 28° C. with the appropriate selection agent. A. tumefaciens cells are pelleted and resuspended in liquid MSPR medium, pH 5.2 containing 40 μM acetosyringone. Callus is added to each tube and maintained under vacuum pressure (approximately 20 mm Hg). Callus is removed from the microfuge tubes and placed in flasks containing liquid MSPR medium in the dark at room temperature for three days. The co-cultivation liquid is replaced with fresh MSPR medium and incubated for three days in the dark. Explants are then washed in liquid MS medium containing 2 g/L of claforan (Hoechst-Roussel) for 30 minutes and blotted dry on sterile filter paper. Explants are transferred to MSPR medium including 300 mg/L of claforan and maintained in the dark at room temperature for a recovery period of approximately four weeks until the callus differentiates. Callus is plated to selection medium for regeneration containing imidazolinone and maintained in the low light for four weeks. Callus is then transferred to MSO medium containing progressively increasing concentrations of the selection agent. Claforan is included in the selection medium to inhibit growth of Agrobacterium. Following eight weeks of selection, callus that contains green shoots is transferred to MSO medium and plants develop. Approximately 20 ng of genomic DNA from each plant is used in a standard PCR reaction to amplify the T-DNA. Plants that have T-DNA in their genome are selected for the subsequent experiment.

The plants are clonally propagated to create identical genotypes. Some are grown in the presence of doxycycline, whereas the exact same genotype is grown in the absence of doxycycline in a paired experiment. The removal of doxycycline activates expression of the recombinase and facilitates the excision of the T-DNA. In the case of plants containing the monocot T-DNA (FIG. 1), the plants grown in the presence of doxycycline have GFP activity, whereas those grown in its absence lack GFP activity. Similarly, seeds of the plants grown in the presence of doxycycline have tolerance of gentamycin, whereas seeds of those grown in its absence are susceptible. This example illustrates the principle of complete excision of all transgenes from the T-DNA in the plant's genome.

To confirm complete excision, DNA is extracted from the transgenic perennial ryegrass plants grown in the presence and absence of doxycycline. DNA samples from those grown in the presence of doxycycline contain a positive PCR band for the region of T-DNA between the recombinase target sites attB and attP, whereas those grown in its absence lack the PCR band, indicating that the T-DNA has been excised from the genomic DNA of these plants. This is confirmed by Southern hybridization. DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer. DNA samples from those grown in the presence of doxycycline contained a positive band for the T-DNA, whereas those grown in its absence lacked the band.

### Example 4

### Excision of T-DNA During Seed Germination

In this example, a promoter that is expressed only during seed germination was used to control expression of the recombinase gene (FIG. 1, pBPS EW151 T-DNA). The promoter region (−3000 bp—+35 bp) of the Arabidopsis thaliana GA4H gene for chromosome 1 (pAtGA4H, SEQ ID NO: 12) was chosen as a representative seed germination promoter for this example because the AtGA4H gene is tightly regulated and is expressed only at the beginning of germination (Yamaguchi et al. 1998 Plant Cell 10:2115-2126), however, any germination specific promoter could fulfill this role.

The self excising cassette form vector pBPS EW151 is validated in planta using Arabidopsis thaliana and perennial ryegrass (Lolium perenne) using standard plant transformation procedures (see above) except that plants are not transformed in the presence of doxycycline. Arabidopsis thaliana is considered as a typical dicotyledonous plant and ryegrass as a typical monocotyledonous plant. T₁ transformed Arabidopsis thaliana plants are selected from the T₁ seed pool in the presence of doxycycline to prevent expression of the recombinase gene during germination. Transgenic seeds of Arabidopsis thaliana or ryegrass are germinated in standard medium supplemented with doxycycline and sampled after the three-leaf stage. As the seed germinates, activation of the recombinase gene is repressed by the doxycycline and no excision of the T-DNA occurs. Shoots and roots of the T₁ plants do contain the T-DNA. Similarly seeds from the plants contain the T-DNA, so that the inheritance of the transgene has been maintained. On the other hand, transgenic seeds of Arabidopsis thaliana or ryegrass are germinated in standard medium (without doxycycline) and sampled after the three-leaf stage. As the seed germinates, the recombinase gene is activated and the genomic DNA is modified by excision of the T-DNA. Shoots and roots of the T₁ plants do not contain the T-DNA. Similarly seeds from the T₁ plants do not contain the T-DNA, so that the inheritance of the transgene has been eliminated.

The transgenic ryegrass plants are produced in the same manner, except that doxycycline is not included in the culture or selection media during transformation or growth of the plants. The putative transgenic ryegrass plants are transferred to the greenhouse. PCR and Southern hybridization are used to select primary (T₀) transgenic plants with single insertions of the T-DNA. Selected plants are cross-pollinated and mature seed is collected. DNA is extracted from the seeds. PCR and Southern hybridization confirm that the T-DNA is present in some of the seeds indicating that the T-DNA is being inherited as a single Mendelian trait. The seeds are germinated in the greenhouse in standard potting soil and sampled after the three-leaf stage. As the seed germinates, the recombinase gene is activated and the genomic DNA is modified by excision of the T-DNA. Shoots and roots of the T₁ plants do not contain the T-DNA. Similarly seeds from the T₁ plants do not contain the T-DNA, so that the inheritance of the transgene has been eliminated. It is however maintained in vegetative propagules of the T₀ plants.

### Example 5

### Excision of T-DNA in Pollen

Any promoter that is expressed during pollen or egg development can also be used to activate the recombinase gene and thereby control the inheritance of the T-DNA. To illustrate this embodiment, the design and construction of the transformation vectors is similar to those in FIG. 1, except that a promoter that is expressed only in pollen or immature pollen is used to control expression of the recombinase gene. In this example, the T-DNA also contains the mutated AHAS gene from maize for resistance to the imidazolinone herbicides. Transgenic ryegrass plants are produced in the same manner as Example 2, except that doxycycline is not included in the culture or selection media during transformation or growth of the plants. The putative primary (T₀) transgenic ryegrass plants are transferred to the greenhouse.

PCR and Southern hybridization are used to select primary (T₀) transgenic plants with single insertions of the T-DNA. The plants are sprayed with an imidazolinone-containing herbicide mixture to select resistant plants. A plant with a single insertion of T-DNA and resistance to the imidazolinone-containing herbicide is vegetatively propagated. It is subsequently cross-pollinated to a non-transgenic plant and seed is produced. The direction of cross-pollination is controlled so that the transgenic plant is either the male or the female parent. The seeds are germinated in the greenhouse in standard potting soil and sprayed with an imidazolinone-containing herbicide. When the transgenic plant is used as the female parent, the T-DNA is present in 50% of the plants as shown by their resistance to the imidazolinone-containing herbicide, indicating that the T-DNA is inherited as a single Mendelian trait. When the transgenic plant is used as the male parent, the T-DNA is excised from the pollen and all of the progeny are susceptible to the imidazolinone-containing herbicide.

### Example 6

### Environmentally Regulated Excision of T-DNA

A promoter that is expressed only following exposure to a specific environmental treatment, in this example, heat shock, can also be used to control expression of a recombinase gene. The design and construction of the transformation vectors is similar to those in FIG. 1, except that a promoter from a heat shock gene is used to control expression of the recombinase gene. The transgenic Arabidopsis and ryegrass plants are produced in the same manner as Example 2, except that doxycycline is not included in the culture or selection media during transformation or growth of the plants.

Seeds of the transgenic Arabidopsis are germinated on selection medium. PCR and Southern hybridization are used to select primary (T₀) transgenic plants with single insertions of the T-DNA. Homozygous transgenic plants are selected and seed produced. The seeds are germinated and half of the seedlings are exposed to a heat shock of 40° C. for a sufficient time to activate expression of the recombinase gene. Seeds are harvested from plants exposed to a heat shock and from control plants not exposed to heat shock. The seed is germinated and the plants are analyzed by PCR and Southern hybridization for the presence of T-DNA. Those grown in the absence of heat shock contain the transgenes in the original T-DNA, whereas in those exposed to the heat shock, the recombinase gene is activated and the genomic DNA is modified by excision of the T-DNA. The putative primary (T₀) transgenic ryegrass plants are transferred to the greenhouse.

PCR and Southern hybridization are used to select primary (T₀) transgenic plants with single insertions of the T-DNA. These plants are vegetatively propagated and divided into paired groups; the plants are defoliated and new shoots regrown from tillers. At the vegetative stage, the shoots of one group are exposed to heat shock conditions, whereas the other control group remains under normal growth conditions. All plants are returned to normal growth conditions and each is subsequently cross-pollinated to a non-transgenic plant and seed is produced. The seeds are germinated in the greenhouse in standard potting soil and sampled after the three-leaf stage. DNA is extracted from the shoots. PCR and Southern hybridization demonstrate that the T-DNA is present in 50% of the plants grown from the seeds of normally grown plants, indicating that the T-DNA is being inherited as a single Mendelian trait. The same tests show that plants grown from the seeds from the heat shocked group of plants lack the T-DNA. In these latter plants, the recombinase gene is activated by the heat shock and the genomic DNA is modified by excision of the T-DNA. The flowers and seeds subsequently produced by these shoots do not contain (or contain very little) T-DNA, so that the inheritance of the transgene has been eliminated (or significantly reduced).

In the perennial ryegrass plants, the inheritance of the T-DNA can be restored provided the crown and roots were not exposed to the heat shock stress. In this instance, defoliation of the ryegrass plants stimulates development of the tillers and new shoots are formed. These new shoots produce flowers and set seed containing the T-DNA because the recombinase was not activated in the cells forming the new shoot. Therefore, inheritance of the T-DNA is restored.

### Example 7

### Chemical Activation of T-DNA Excision

A binary vector is constructed that contains the φC31int^{INT} or φC31int*^{INT} recombinase gene controlled by a transactivator's target promoter, a chemically activated transactivator gene controlled by a constitutive promoter. Self excising recombinase cassettes are constructed similar to those in FIG. 1. These self excising recombinase cassettes are validated in planta using Arabidopsis thaliana and perennial ryegrass (Lolium perenne) and standard plant transformation procedures (see above). Plants are transformed and selected in the absence of induction. Upon establishment of stable transformed lines, the T-DNA can be excised at will by exposing the plant to the chemical. With a chemically activated transactivator, the entire T-DNA may be removed prior to export to countries that do not prefer or permit genetically modified plants or prior to export to nations with uncertain protection of intellectual property. In all of these cases, excision is activated by treatment with a chemical that activates the chimeric transactivator, i.e. the glucocorticoid or ecdysone receptor ligands, depending upon the activation system chosen.

### Example 8

### Excision of T-DNA Using a Chemically Regulated Ligand Binding Domain

In this example, a promoter that is expressed only during seed germination is used to directly control expression of the recombinase gene. The promoter region (−3000 bp—+35 bp) of the Arabidopsis thaliana GA4H gene for chromosome 1 (pAtGA4H, SEQ ID NO:12) was chosen as a representative seed germination promoter (FIG. 2). Chemical regulation is maintained by a recombinase-Ligand Binding Domain (LBD) fusion protein. For the proper repressive regulation to occur, the LBD must be regulated such that ligand binding triggers relocation of the fusion protein into the nucleus. The LBD of the Drosophila melanogaster methoprene-tolerant (metLBD) gene (PCT WO98/46724) was chosen as a representative LBD.

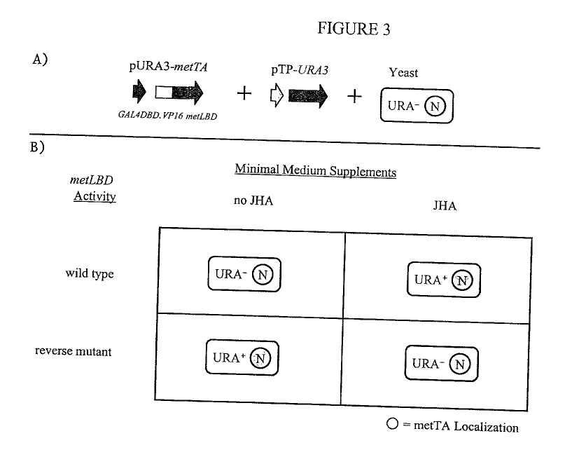

In the present invention, a reverse regulated mutation is desirable to achieve chemically repressed regulation. Such a reverse regulated mutation can be identified using a metLBD based gene switch in yeast (FIG. 3). A metLBD based gene switch can be derived by translationally fusing a DNA binding domain (DBD, e.g. GAL4 DBD) with an acidic activator domain (AD, e.g. VP16) and the metLBD. This metLBD derived transactivator (metTA) would be used with a chimeric target promoter (pTP) in which several DNA binding domain target sites are placed up stream (5′) of a minimal promoter. A screen for reverse activity can then be constructed in an URA⁻ yeast strain transformed (nuclear) with a construct containing a URA3 gene transcriptionally regulated by pTP (pTP-URA). Then, using another construct containing the metTA gene controlled by a constitutive promoter like the URA3 promoter (pURA-metTA).

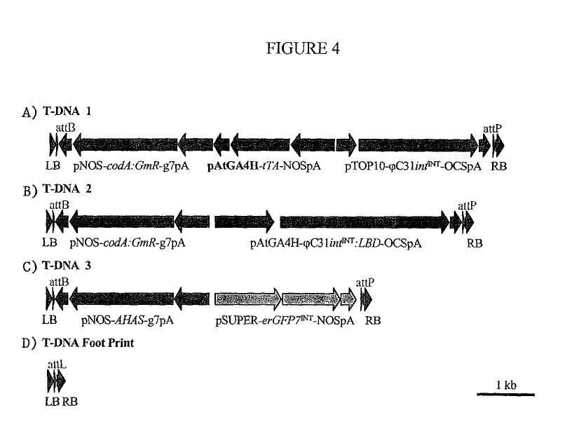

When both pURA-metTA and pTP-URA are present within the nucleus of a eukaryotic cell, the presence of juvenile hormone analogs (JHA), like methoprene, will induce transcription from pTP and result in a URA⁺ phenotype; however in the absence of JHA, no transcription will occur and will result in a URA⁻ phenotype (FIG. 3). The URA⁺ phenotype can be screened for by the survival on minimal medium, and the URA⁻ phenotype can be screened for on complex medium supplemented with 5-fluorotic acid (5FOA). Since either phenotype can be selected, a simple screen can be conducted. First, targeted or random mutation of the metLBD is preformed in pURA-metTA. Second, the mutants are transformed into an URA⁻ yeast harboring nuclear pTA-URA. Third, the yeast are selected for the URA⁺ phenotype in the absence of JHA ligand. Forth, the resulting URA⁺ yeast are screened for a URA⁻ phenotype in the presence of JHA ligand. Finally, the metLBD gene fragments of any survivors are sub-cloned and analyzed further. True reverse metTA mutants, hereafter designated rmetTA, should survive the Third and Forth steps (above) when re-tested and could serve as the LBD in FIG. 4.

The self excising T-DNA vector (FIG. 2) is validated in planta using Arabidopsis thaliana and perennial ryegrass (Lolium perenne) using standard plant transformation procedures (see above) except that plants are not transformed in the presence of doxycycline. Arabidopsis thaliana is considered as a typical dicotyledonous plant and ryegrass as a typical monocotyledonous plant. T₁ transformed Arabidopsis thaliana plants are selected from the T₁ seed pool in the presence of doxycycline to prevent expression of the recombinase gene during germination. Then, transgenic seeds of Arabidopsis thaliana or ryegrass are germinated in standard medium supplemented with doxycycline and sampled after the three-leaf stage. As the seed germinates, activation of the recombinase gene is repressed by the chosen ligand and no excision of the T-DNA occurs. Shoots and roots of the T₁ plants do contain the T-DNA. Similarly seeds from the plants contain the T-DNA, so that the inheritance of the transgene has been maintained. On the other hand, transgenic seeds of Arabidopsis thaliana or ryegrass are germinated in standard medium (without ligand) and sampled after the three-leaf stage. As the seed germinates, the recombinase gene is activated and the genomic DNA is modified by excision of the T-DNA. Shoots and roots of the T₁ plants do not contain the T-DNA. Similarly seeds from the T₁ plants do not contain (or contain very little) T-DNA, so that the inheritance of the transgene has been eliminated (or significantly reduced).

### Example 9

### Excision of Distal T-DNAs by a Self Excising Cassette

In the previous examples, regulated self excision cassettes for purposes of transgene containment were described. Frequently in modem biotechnological applications numerous genes are required to achieve an economically viable crop improvement. Therefore, several trait genes often need to be stacked into one plant with multiple transformations. In this example, the agronomically important trait gene(s) are located on a separate T-DNA from the self excising cassette to allow for easier gene stacking.

In this example, a promoter that is expressed only during seed germination is used to directly control expression of the recombinase gene (FIG. 4). The promoter region (−3000 bp—+35 bp) of the Arabidopsis thaliana GA4H gene for chromosome 1 (pAtGA4H, SEQ ID NO:12) was chosen as a representative seed germination promoter for this example. Chemical regulation can be maintained by either a transcriptionally regulated gene switch (FIG. 1 pBPS EW151 T-DNA or FIG. 4 T-DNA 1) or by a recombinase-LBD fusion protein (FIG. 2 T-DNA or FIG. 4 T-DNA2). Regardless of the regulatory method, these constructs can also facilitate the excision of unlinked T-DNA constructs as well (FIG. 4 T-DNA 3). A random order of excision might result in as much as 50% retention of the secondary target T-DNA; however, since genomic location alters the accessibility of recombination sites, in practice most or all of the target T-DNA might ordinarily remain after excision of the primary, self-excising T-DNA. Such an improper excision sequence can be prevented by two mechanisms. First, a simple screen for the transgenes that have the desired excision kinetics can be performed among a population of different insertion events. Secondly, the excision kinetics can be favorably altered by utilizing different recombination sites. Minimal or non-optimal target sites (target site fragments or those containing point mutations) should be used for the self-excising transgenes and full length or optimal recombination sites should be utilized for the target transgenes.

The target-excising cassette from T-DNA 3 (FIG. 4) is validated in planta using Arabidopsis thaliana and perennial ryegrass (Lolium perenne) using standard plant transformation procedures (see above) except that plants are not transformed in the presence of doxycycline. T₁ transformed plants are selected and crossed with a self-excising cassette T-DNA (FIGS. 1, 2, or 4). The resulting F₁ transgenic seeds of Arabidopsis thaliana or ryegrass are germinated in standard medium supplemented with doxycycline and sampled after the three-leaf stage. As the seed germinates, activation of the recombinase gene is repressed by the chosen ligand and no excision of the T-DNA occurs. (Dual selection can be applied with the doxycycline to assist in the establishment of homozygous lines.) Shoots and roots of the F₁ plants contain the T-DNA. Similarly seeds from the plants contain the T-DNA, so that the inheritance of the transgene has been maintained. On the other hand, transgenic F₁ seeds of Arabidopsis thaliana or ryegrass are germinated in standard medium (without ligand) and sampled after the three-leaf stage. As the seed germinates, the recombinase gene is activated and the genomic DNA is modified by excision of the T-DNA(s). Shoots and roots of the T₁ plants do not contain the T-DNA(s). Similarly seeds from the T₁ plants do not contain the T-DNA, so that the inheritance of the transgene has been eliminated. Southern or PCR analysis can then estimate which loci combined to best and eliminated the greatest percentage of the target T-DNA prior to loss of the recombinase gene itself.

### Example 10

### Excision of T-DNA in Gymnosperm Pollen

Any promoter that is expressed during pollen or egg development can be used to activate a recombinase gene (see Example 5) or a chemically regulatable recombinase gene (see Example 8). The recombinase gene can be chemically regulatable through a gene switch such as chemically regulatable promoter or through a ligand binding domain of a nuclear receptor. FIG. 5 illustrates the use of all three systems in one construct, as it contains a developmentally regulated promoter, a gene switch and a recombinase/nuclear receptor fusion polynucleotide. The gene switch is operationally linked to a promoter that is active only in pollen, immature pollen, male cone or tapetum. Specifically for this example, the tapetum specific A9 promoter of Arabidopsis thaliana (Paul et al., 1992 Plant Molecular Biology 19:611-622), Brassica napus (Turgut et al., 1994 Plant Molecular Biology 24:97-104) or homologous promoter from Pinus radiata (Walden et al., 1999 Plant Physiology 121:1103-1116) is used. A promoter which is controlled by the gene switch transactivator is used to control expression of the recombinase gene (FIG. 5). Therefore, in this example, chemical application (e.g. methoprene) represses both transcriptional expression of the φC31int^{INT}:LBD gene and the activity by re-localization of φC31INT:LBD protein to the cytosol.

White pine is chosen as a representative gymnosperm tree. Stable genetic transformation of white pine (Pinus strobus) is performed by cocultivation of embryogenic tissues with Agrobacterium according to Levée et al. (1999 Molecular Breeding 5:429-440). The kanamycin resistant putative primary (T₀) transgenic white pine plantlets are transferred to the greenhouse. PCR and Southern hybridization are used to select primary (T₀) transgenic plants with single insertions of the T-DNA. A plant with a single insertion of T-DNA maybe vegetatively propagated through tissue culture or grafting. The resulting transgenic trees are grown to maturity and seeds produced. The direction of cross-pollination is controlled so that the transgenic plant is either the male or the female parent. The seeds are germinated in the greenhouse in standard potting soil and watered with selective agent (e.g. antibiotic such as kanamycin or herbicide such as glyphosate, phosphinothricen, imidazoinone, ect.). When the transgenic plant is used as the female parent, the T-DNA is present in 50% of the plants as shown by their resistance to kanamycin, indicating that the T-DNA is inherited as a single Mendelian trait. When the transgenic plant is used as the male parent, the T-DNA is excised from the pollen and all of the progeny are susceptible to the selective agent. However, when crosses occur in the presence of the repressive chemical (e.g. methoprene), then all the traits will be inherited as a single Mendelian trait through both the male and female gametes.

The use of a tapetum- or pollen-specific promoter operationally linked to the recombinase gene can be used to prevent the transmission of T-DNA through pollen. This will contain the T-DNA within the female and reduce outcrossing to wild groves in forestry species that are naturally cross-pollinating gymnosperms including, pines, cedars and eucalyptus. The tapetum promoter is simply an example of a promoter that is regulated in a specific developmental manner. By the careful choice of promoter, the recombinase transgene can be activated in specific tissue and at a specific stage of development. Chimeric plants can therefore be created if promoters that are active in vegetative tissues are used.

We claim:

1. An isolated excisable polynucleotide comprising, a desired trait polynucleotide and a recombinase polynucleotide operably linked to a promoter, all flanked by a pair of directly oriented recombination sites, wherein the recombinase activity is regulatable.

2. The isolated polynucleotide of claim 1, wherein the recombinase is selected from the group consisting of, φC31, FLP, CRE, resolvase, SSV1-encoded integrase, R, Gin and transposase.

3. The isolated polynucleotide of claim 1, wherein the recombinase is φC31.

4. The isolated polynucleotide of claim 3, wherein the φC31 recombinase polynucleotide comprises an intron.

5. The isolated polynucleotide of claim 3, wherein the φC31 recombinase polynucleotide comprises a sequence as shown in SEQ ID NO:9 or SEQ ID NO:10.

6. The isolated polynucleotide of claim 1, further comprising a selectable marker polynucleotide that is also flanked by the pair of recombination sites.

7. The isolated polynucleotide of 1, wherein the promoter is active in a plant cell, but inactive in a prokaryote.

8. The isolated polynucleotide of claim 1, wherein the promoter is developmentally regulated.

9. The isolated polynucleotide of claim 8, wherein the promoter is selected from the group consisting of a seed-preferred, leaf-preferred, root-preferred, pollen-preferred, egg-preferred promoter, germination-preferred, meristem-preferred, tuber-preferred, ovule-preferred and anther-preferred.

10. The isolated polynucleotide of claim 8, wherein the promoter is a seed-preferred promoter.

11. The isolated polynucleotide of claim 8, wherein the promoter is a germination-preferred promoter.

12. The isolated polynucleotide of claim 8, wherein the promoter is a pollen-preferred promoter.

13. The isolated polynucleotide of claim 1, wherein the promoter is environmentally regulated.

14. The isolated polynucleotide of claim 13, wherein the promoter is regulated by an environmental factor or condition selected from the group consisting of, heat-shock, pathogen attack, anaerobic conditions, elevated temperature, decreased temperature, the presence of light and a chemical factor.

15. The isolated polynucleotide of claim 13, wherein the promoter is a heat shock activated promoter.

16. The isolated polynucleotide of claim 13, wherein the recombinase activity is repressible.

17. The isolated polynucleotide of claim 16, wherein the promoter is repressed by a chemical.

18. The isolated polynucleotide of claim 17, further comprising a chemically activated transactivator that represses the promoter, wherein in the transactivator is also flanked by the recombination sites.

19. The isolated polynucleotide of claim 16, wherein the recombinase polynucleotide is operably linked to a chemical ligand receptor domain of a nuclear receptor.

20. The isolated polynucleotide of claim 19, further comprising a chemically activated transactivator that represses the promoter, wherein in the transactivator is also flanked by the recombination sites.

21. A plant cell comprising the excisable polynucleotide of any of claims 1-20.

22. A plant comprising the plant cell of claim 21.

23. The plant of claim 22, wherein the plant is a dicot.

24. The plant of claim 22, wherein the plant is a monocot.

25. A seed produced by the plant of claim 22.

26. The seed of claim 25, further comprising a chemical coating, wherein the chemical represses expression of the recombinase polynucleotide or represses the activity of a recombinase polypeptide encoded by the recombinase polynucleotide.

27. A tree comprising the excisable polynucleotide of claim 1.

28. An isolated φC31 recombinase polynucleotide comprising an intron.

29. The isolated φC31 recombinase polynucleotide of claim 28, wherein the polynucleotide comprises a sequence as shown in SEQ ID NO:9 or SEQ ID NO:10.

30. A method of producing a transgenic plant containing an isolated excisable polynucleotide comprising,
a. introducing into a plant cell the isolated excisable polynucleotide, wherein the excisable polynucleotide comprises a desired trait polynucleotide and a recombinase polynucleotide operably linked to a promoter, all flanked by a pair of recombination sites in direct orientation, wherein the recombinase activity is regulatable; and
b. generating from the plant cell the transgenic plant.

31. The method of claim 30, wherein the recombinase is selected from the group consisting of, φC31, FLP, CRE, resolvase, SSV1-encoded integrase, R, Gin and transposase.

32. The method of claim 30, wherein the recombinase is φC31.

33. The method of claim 32, wherein the φC31 recombinase polynucleotide comprises an intron.

34. The method of claim 32, wherein the φC31 recombinase polynucleotide comprises a sequence as shown in SEQ ID NO:9 or SEQ ID NO:10.

35. The method of claim 30, wherein the excisable polynucleotide further comprises a selectable marker polynucleotide that is also flanked by the pair of recombination sites.

36. The method of claim 30, wherein the promoter is active in a plant cell, but inactive in a prokaryote.

37. The method of claim 30, wherein the promoter is developmentally regulated.

38. The method of claim 37, wherein the promoter is selected from the group consisting of a seed-preferred, leaf-preferred, root-preferred, pollen-preferred, egg-preferred promoter, germination-preferred, meristem-preferred, tuber-preferred, ovule-preferred and anther-preferred.

39. The method of claim 37, wherein the promoter is a seed-preferred promoter.

40. The method of claim 37, wherein the promoter is a germination-preferred promoter.

41. The method of claim 37, wherein the promoter is a pollen-preferred promoter.

42. The method of claim 30, wherein the promoter is environmentally regulated.

43. The method of claim 42, wherein the promoter is regulated by an environmental factor or condition selected from the group consisting of, heat-shock, pathogen attack, anaerobic conditions, elevated temperature, decreased temperature, the presence of light and a chemical factor.

44. The method of claim 42, wherein the promoter is a heat shock activated promoter.

45. The isolated polynucleotide of claim 42, wherein the recombinase activity is repressible.

46. The isolated polynucleotide of claim 45, wherein the promoter is repressed by a chemical.

47. The isolated polynucleotide of claim 46, further comprising a chemically activated transactivator that represses the promoter, wherein in the transactivator is also flanked by the recombination sites.

48. The isolated polynucleotide of claim 45, wherein the recombinase polynucleotide is operably linked to a chemical ligand receptor domain of a nuclear receptor.

49. A method of expressing an excisable transgenic trait in a plant, comprising
a. providing a plant comprising an excisable polynucleotide, wherein the excisable polynucleotide comprises a desired trait polynucleotide and a recombinase polynucleotide operably linked to a promoter, all flanked by a pair of recombination sites in direct orientation; and
b. exposing the plant to a condition or factor that represses activity of the recombinase.

50. The method of claim 49, wherein the recombinase is selected from the group consisting of, φC31, FLP, CRE, resolvase, SSV1-encoded integrase, R, Gin and transposase.

51. The method of claim 49, wherein the recombinase is φC31.

52. The method of claim 51, wherein the φC31 recombinase polynucleotide comprises an intron.

53. The method of claim 51, wherein the φC31 recombinase polynucleotide comprises a sequence as shown in SEQ ID NO:9 or SEQ ID NO:10.

54. The method of claim 49, wherein the promoter is repressed by an environmental factor or condition selected from the group consisting of, a chemical, heat-shock, pathogen attack, anaerobic conditions, elevated temperature, decreased temperature and the presence of light.

55. The method of claim 49, wherein factor is a chemical.

56. The method of claim 55, wherein the excisable polynucleotide further comprises a chemically activated transactivator that represses the promoter, wherein in the transactivator is also flanked by the recombination sites.

57. The method of claim 55, wherein the recombinase polynucleotide is operably linked to a chemical ligand receptor domain of a nuclear receptor.

58. A method of gene stacking in a cell comprising:
a. introducing into the cell a first excisable polynucleotide comprising a first desired trait polynucleotide and a recombinase polynucleotide operably linked to a repressible promoter, all flanked by a first pair of recombination sites in direct orientation;
b. introducing into the cell a second excisable polynucleotide comprising a second desired trait polynucleotide operably linked to a promoter, all flanked by a second pair of recombination sites in direct orientation, wherein the second pair of recombination sites are more efficiently excised than the first pair of recombination sites; and
c. culturing the cell under a condition that represses activity of the recombinase.

59. The method of claim 58, wherein the first recombination sites contain one or more point mutations.

60. The method of claim 58, wherein the first recombination sites contain one or more deletions.
